SEQUENCE LISTING (15 sequences)

SEQ ID NO: 1 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - protein, 408 aa, Homo sapiens
MIPGNRMLMVVLLCQVLLGGASHASLIPETGKKKVAEIQGHAGGRRSGQSHELLRDFEATLLQMFGLRRRPQPSKSAVIPDYMRDLYRLQSGEEEEEQIHSTGLEYPERPASRANTVRSFHHEEHLENIPGTSENSAFRFLFNLSSIPENEAISSAELRLFREQVDQGPDWERGFHRINIYEVMKPPAEVVPGHLITRLLDTRLVHHNVTRWETFDVSPAVLRWTREKQPNYGLAIEVTHLHQTRTHQGQHVRISRSLPQGSGNWAQLRPLLVTFGHDGRGHALTRRRRAKRSPKHHSQRARKKNKNCRRHSLYVDFSDVGWNDWIVAPPGYQAFYCHGDCPFPLADHLNSTNHAIVQTLVNSVNSSIPKACCVPTELSAISMLYLDEYDKVVLKNYQEMVVEGCGCR

SEQ ID NO: 3 - protein, 454 aa, Homo sapiens
MHLTVFLLKGIVGFLWSCWVLVGYAKGGLGDNHVHSSFIYRRLRNHERREIQREILSILGLPHRPRPFSPGKQASSAPLFMLDLYNAMTNEENPEESEYSVRASLAEETRGARKGYPASPNGYPRRIQLSRTTPLTTQSPPLASLHDTNFLNDADMVMSFVNLVERDKDFSHQRRHYKEFRFDLTQIPHGEAVTAAEFRIYKDRSNNRFENETIKISIYQIIKEYTNRDADLFLLDTRKAQALDVGWLVFDITVTSNHWVINPQNNLGLQLCAETGDGRSINVKSAGLVGRQGPQSKQPFMVAFFKASEVLLRSVRAANKRKNQNRNKSSSHQDSSRMSSVGDYNTSEQKQACKKHELYVSFRDLGWQDWIIAPEGYAAFYCDGECSFPLNAHMNATNHAIVQTLVHLMFPDHVPKPCCAPTKLNAISVLYFDDSSNVILKKYRNMVVRSCGCH

SEQ ID NO: 4 - protein, 513 aa, Homo sapiens
MPGLGRRAQWLCWWWGLLCSCCGPPPLRPPLPAAAAAAAGGQLLGDGGSPGRTEQPPPSPQSSSGFLYRRLKTQEKREMQKEILSVLGLPHRPRPLHGLQQPQPPALRQQEEQQQQQQLPRGEPPPGRLKSAPLFMLDLYNALSADNDEDGASEGERQQSWPHEAASSSQRRQPPPGAAHPLNRKSLLAPGSGSGGASPLTSAQDSAFLNDADMVMSFVNLVEYDKEFSPRQRHHKEFKFNLSQIPEGEVVTAAEFRIYKDCVMGSFKNQTFLISIYQVLQEHQHRDSDLFLLDTRVVWASEEGWLEFDITATSNLWVVTPQHNMGLQLSVVTRDGVHVHPRAAGLVGRDGPYDKQPFMVAFFKVSEVHVRTTRSASSRRRQQSRNRSTQSQDVARVSSASDYNSSELKTACRKHELYVSFQDLGWQDWIIAPKGYAANYCDGECSFPLNAHMNATNHAIVQTLVHLMNPEYVPKPCCAPTKLNAISVLYFDDNSNVILKKYRNMVVRACGCH

SEQ ID NO: 5 - protein, 431 aa, Homo sapiens
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 6 - protein, 16 aa, Artificial Sequence
  synthesized oligopeptide
CIVNSSDDFLCKKYRS

SEQ ID NO: 7 - protein, 16 aa, Artificial Sequence
  synthesized oligopeptide
CYFNDSSQVLCKRYRS

SEQ ID NO: 8 - DNA, 24 bp, Artificial Sequence
  Oligonucleotide
tatctctgctcgtgtgtttgggca

SEQ ID NO: 9 - DNA, 24 bp, Artificial Sequence
  Oligonucleotide
attgatgtagtgtcgcagagcgga

SEQ ID NO: 10 - DNA, 22 bp, Artificial Sequence
  oligonucleotide
cggccccaggaacagcagcagt

SEQ ID NO: 11 - DNA, 19 bp, Artificial Sequence
  oligonucleotide
gggcccgtcgtccttctcc

SEQ ID NO: 12 - DNA, 21 bp, Artificial Sequence
  oligonucleotide
ttgtgttctgctgttggcact

SEQ ID NO: 13 - DNA, 21 bp, Artificial Sequence
  oligonucleotide
agcaaaaggcattgaagaagc

SEQ ID NO: 14 - DNA, 21 bp, Artificial Sequence
  oligonucleotide
tgctaatgatgatggctttcc

SEQ ID NO: 15 - DNA, 20 bp, Artificial Sequence
  oligonucleotide
ttcacagtggtcctcgttcc

(19) United States
(12) Patent Application Publication
(10) Pub. No.: US 2011/0046047 A1
(43) Pub. Date: Feb. 24, 2011

(54) Bone Morphogenetic Proteins for Appetite Control

(75) Inventors: Yu-Hua Tseng, Newton, MA (US); Efthymia Kokkotou, Boston, MA (US)

Correspondence Address: FISH & RICHARDSON P.C. (BO), P.O. BOX 1022, MINNEAPOLIS, MN, 55440-1022, US

(73) Assignee: JOSLIN DIABETS CENTER, INC.

(21) Appl. No.: 12/784,228

(22) Filed: May 20, 2010

Related U.S. Application Data
(60) Provisional application No. 61/179,977, filed on May 20, 2009.

(51) Int. Cl. A61K 38/18; A61P 1/00

(52) U.S. Cl. 514/4.9

(57) ABSTRACT

Methods of decreasing appetite for food intake by administering bone morphogenetic proteins (BMPs), e.g., BMP7, or agonists/peptidomimetics thereof.

### CLAIM OF PRIORITY

This application claims the benefit of U.S. Patent Application Ser. No. 61/179,977, filed on May 20, 2009, the entire contents of which are hereby incorporated by reference.

### FEDERALLY SPONSORED RESEARCH OR DEVELOPMENT

This invention was made with Government support under Grant No. R01 DK077097 awarded by the National Institutes of Health. The Government has certain rights in the invention.

### TECHNICAL FIELD

This invention relates to composition and methods for modulating (e.g., suppressing) appetite for food intake using bone morphogenetic protein.

### BACKGROUND

Overeating, or an energy intake that exceeds expenditure, causes weight gain because excess energy is stored in mammals (e.g., humans) as adipose tissue or fat. Prolonged overeating results in obesity. Obesity contributes to or is causative in a number of potentially serious health problems. Such health problems include, for example, type II diabetes, insulin insensitivity, cancer, cardiovascular disease, metabolic syndrome, liver disease, osteoarthritis, and depression. The incidence of obesity is high and increasing, largely due to modern day environmental factors and sedentary life styles. Therapies to reduce or prevent the occurrence of obesity, for example, by reducing energy intake/overeating are required.

### SUMMARY

The present disclosure is based, at least in part, on the discovery that bone morphogenetic protein (BMP), e.g., BMP7, can be used to modulate (e.g., decrease) appetite for food intake in a subject.

Thus, the invention provides methods for suppressing appetite for food intake in a subject, the method comprising increasing bone morphogenic protein 7 (BMP7) expression, activity, or signaling in the central nervous system of the subject, by administering one or more BMP7 agents to the subject. In some embodiments, the methods also include evaluating food intake (e.g., daily food intake, e.g., daily caloric intake, or nutritional content of food consumed) at a first time (e.g., before administration of the BMP7 agent) to establish a first value; evaluating food intake at a second time after administration of one or more doses of the BMP7 agent to establish a second value; and comparing the first value and the second value to detect an effect of the BMP7 agent on appetite. A decrease in food intake from the first value to the second value indicates the effect of the BMP7 on appetite in the subject. In some embodiments, the methods further, include adjusting the dose of the BMP7 agent to optimize the effect on appetite in the subject (e.g., increase the dose to decrease appetite, or decrease the dose to increase appetite, optimally to approach a desired food intake value, e.g., a value that represents a level of intake that would enable the subject to lose weight, or reach or maintain a desired weight).

In some embodiments, the BMP7 agent is administered systemically, e.g., parenterally.

In some embodiments, the one or more BMP7 agents comprises a BMP7 polypeptide, e.g., a polypeptide with at least 95% identity to amino acids 293 431 of SEQ ID NO:5. In some embodiments, the BMP7 agent comprises a BMP7 peptide or peptide mimetic. In some embodiments, the one or more BMP7 agents comprises a nucleic acid molecule encoding a BMP7 polypeptide, e.g., a nucleic acid molecule that encodes a BMP7 polypeptide with at least 95% identity to amino acids 293 431 of SEQ ID NO:5.

In some embodiments, the subjects are selected on the basis that the subject is in need of appetite suppression. In some embodiments, the methods include selecting a subject who has a body mass index (BMI) of at least 25 (e.g., the subject is selected on the basis that the subject has a BMI of over 25). In some embodiments, the subject has a BMI of at least 30.

In some embodiments, the methods include selecting a subject who has a daily food intake above a selected level (e.g., above a level that would allow the subject to reach or maintain a desired weight).

### DEFINITIONS

As used herein, the term “subject” refers to an animal, human or non-human, rodent or non-rodent, to whom treatment according to the methods of the present invention is provided. Veterinary and non-veterinary applications are contemplated. The term includes, but is not limited to, mammals, e.g., humans, other primates, pigs, rodents such as mice and rats, rabbits, guinea pigs, hamsters, cows, horses, cats, dogs, sheep and goats. Typical subjects include humans, farm animals, and domestic pets such as cats and dogs. In some embodiments, the subject is a mammal. In some embodiments, the subject is a human subject, e.g., an obese human subject. In some embodiments, the subject is a non-human mammal, e.g., an experimental animal, a companion animal, or a food animal, e.g., a cow, pig, or sheep that is raised for food.

The terms “effective amount” and “effective to treat,” as used herein, refer to an amount or a concentration of one or more of the compositions described herein utilized for a period of time (including acute or chronic administration and periodic or continuous administration) that is effective within the context of its administration for causing an intended effect or physiological outcome. In some embodiments, the desired treatment outcome is a reduction in food intake, e.g., a reduction in the number of calories consumed per day. The reduction can be, e.g., 10%, 15%, 20%, 30%, 40%, 50%, 60%, or more, of the subject's food or caloric intake. The reduction can be sufficient to result in weight loss.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A is an image showing lac Z staining (exemplified by arrows) of the rostal hypothalamus (coronal sections) in BMP7 heterozygous mice carrying a beta galactosidase reporter allele in the BMP7 locus.

FIG. 1B is an image showing BMPR2 expression detected by in-situ hybridization in the rostal and caudal hypothalamus. Antisense shows specific staining. Sense shows non-specific (background) staining.

FIGS. 2A-2D are bar graphs showing changes in expression of BMP7 and ALK2 in second-order hypothalamic nuclei of mice fed a 60% high fat diet for the indicated time period, as assessed by quantitative PCR. Open bars, low fat diet; Filled bars, high fat diet.

FIG. 3A is a bar graph showing food consumption for C57BL/6 mice over 4 hours following intracerebroventricular (ICV) administration of control (vehicle), recombinant human BMP7, or leptin.

FIGS. 3B-3G are images showing α-MSH expression in rostral and claudal hypothalamus 60 minutes after i.c.v. injection with either vehicle, BMP7, or lectin.

FIGS. 4A-4B are bar graphs showing mRNA expression of several hypothalamic neuropeptides in vehicle, BMP7 and leptin expressing mice, showing that central administration of BMP7 decreased food consumption in C57BL/6 mice. Expression of neuropeptides at 2 hours (4A) and 4 hours (4B) after ICV administration of vehicle, BMP7 or leptin, is shown.

FIG. 5 is an image showing Western blots to detect protein phosphorylation in mice 30, 60, and 120 minutes post ICV administration of BMP7.

FIG. 6 is a bar graph showing food consumption in control or BMP7 treated DIO mice in the absence or presence of rapamycin.

FIG. 7 is a line graph showing plasma BMP-7 levels in C57BL/6 mice after tail vein injection with adenoviruses expressing BMP-7 or LacZ.

FIG. 8A is a bar graph showing a reduction in food intake in BMP7 (Ad-BMP7) treated mice as compared to control (Ad-LacZ) DIO mice.

FIG. 8B is a bar graph showing a BMP7 dependent reduction of body weight in diet-induced obese (DIO) mice.

FIG. 8C is a bar graph showing a BMP7-dependent reduction in body weight in control, pair-fed, and BMP7 treated DIO mice.

FIG. 8D is a bar graph showing a BMP7-dependent increase in oxygen consumption in DIO mice, as assessed using the CLAMS system.

FIG. 9 is a line graph showing body weight up to 14 days post treatment with BMP7 (Ad-BMP7) or control (Ad-LacZ) in DIO mice.

FIG. 10A is a bar graph showing lean mass and muscle fat mass in BMP7 and control treated DIO mice.

FIG. 10B is a bar graph showing leptin levels in BMP7 and control treated DIO mice.

FIG. 10C is a line graph showing insulin tolerance in control or BMP7 treated DIO mice, as assessed using the insulin tolerance test (ITT).

FIG. 10D is a line graph showing glucose tolerance in control or BMP7 treated DIO mice.

FIGS. 10E-F are photomicrographs showing tissue morphology in fat (10E) and liver (10F) in control (Ad-LacZ, top panels) and BMP7 (Ad-BMP7, lower panels) treated DIO mice.

FIG. 11A is a bar graph showing food intake levels over a 4 hour period for wild type and BMP7 treated Ob/Ob mice.

FIG. 11B is a bar graph showing cumulative food intake for Ob/Ob mice treated with control or BMP7.

FIG. 11C is a line graph showing body weight up to 14 days post treatment with BMP7 (Ad-BMP7) or control (Ad-LacZ) in Ob/Ob mice.

FIG. 11D is a bar graph showing body weight of Ob/Ob mice 12 days after treatment with BMP7 (Ad-BMP7) or control (Ad-LacZ).

### DETAILED DESCRIPTION

The present disclosure provides, inter alia, compositions and methods for modulating (e.g., suppressing) appetite in a subject. More specifically, the present disclosure provides that BMP (e.g., BMP7) can be used to suppress appetite and, thereby, promote weight loss in a subject.

Appetite is the desire to eat food and is manifested as hunger. Appetite normally serves to regulate adequate energy intake to maintain metabolic needs. Ideally, a subject's appetite should correspond with their metabolic needs. Appetite that exceeds metabolic needs can result in overeating and weight gain associated therewith. Decreased desire to eat (e.g., suppressed appetite) is termed anorexia. Conversely, increased desire to eat (e.g., elevated or enhanced appetite) is termed polyphagia or hyperphagia. Dysregulation of appetite contributes to anorexia nervosa, bulimia nervosa, cachexia, overeating, and binge eating disorder.

Appetite is normally regulated by the digestive tract, adipose tissue, numerous hormones (e.g., leptin), and the central and peripheral nervous systems. The hypothalamus is the region of the brain most closely associated with appetite regulation.

The hypothalamus receives signals from the periphery, including vagus and sympathetic nerve enervation, circulating nutrients (such as glucose and fatty acids) and hormones (such as adipocyte-derived leptin and gut-derived ghrelin). Such signals act at the arcuate nucleus of the hypothalamus and trigger release/expression of anorexigenic pro-opiomelanocortin (POMC) neurons and orexigenic neuropeptide Y/agouti-related protein (NPY/AgRP) neurons. These neurons serve to act on second-order neurons in hypothalamic areas such as the paraventricular nucleus (PVN), ventromedial hypothalamus (VMH), dorsomedial hypothalamus (DMH) and lateral hypothalamus (LH). These second-order hypothalamic neurons, which include contain orexin and melanocortin signaling components, subsequently signal to extra-hypothalamic regions, and thereby exert regulatory effects on appetite and energy expenditure (reviewed in Ahima and Antwi, Endocrinol. Metab. Clin. North America, 37:811-823 (2008) and Woods and D'Alessio, J. Clin. Endocrinol. Metab., 93:S37-S50 (2008)).

Leptin and insulin activate POMC neurons and inhibit NPY/AgRP neurons. Accordingly, leptin and insulin act as anorexigenic factors which decrease appetite and food intake. Neurotrophic factors such as Brain Derived Neurotrophic Factor (BDNF) and Ciliary Neurotrophic Factor (CNTF) are also anorexigenic factors (Lebrun et al., Auton. Neurosci., 126-127:30-38 (2006) and Vacher et al., FEBBS lett., 582:3832-3838 (2008)).

BMPs are members of the transforming growth factor+superfamily that are most commonly known for their role in key steps of embryogenesis and morphogenesis. More recently, however, a role for BMPs in energy metabolism has been described (Tseng et al., Nature 454:1000-1004 (2008); Schulz et al., Diabetes 58 [Supplement 1], Abstract (2009); Tobin and Celeste, Drug Disc. Today, 11:405-411 (2006); and Ballard et al., Dev. Biol., 337:375-385 (2010)). Consistent with this role, BMPs and their receptors are reportedly expressed in tissues involved in the control of energy homeostasis such as the brain, gut, and adipose tissue (Charytoniuk et al., Neuroscience, 100:33-43 (2000)).

BMP7 belongs to the TGFβ superfamily and was initially identified as a 431 amino acid precursor, based on its ability to promote bone and cartilage growth (Ozkaynak et al., EMBO J., 9:2085-2093 (1990)). More recently, BMP7 has been shown to be involved in numerous other physiological processes, such as, for example: overall embryonic development and morphogenesis by regulating growth, apoptosis and differentiation of a variety of cell types, including epithelial, mesenchymal and neuronal (Oxburgh, Curr. Genomics, 10:223-230 (2009); Bani-Yaghoub et al., Biochem. Cell. Biol., 86:361-369 (2008)); adult neurogenesis (Lein et al., Neuron, 15:597-605 (1995); Chou et al., J. Neurol Sci., 240:21-29 (2006); and Bani-Yaghoub et al., supra (2008)); and the development of neuronal plasticity, in brain areas such as the olfactory system (e.g., Peretto et al., J. Comp. Neurol., 451:267-278 (2002)) and hypothalamus (e.g., Ohyama et al., Development, 132:5185-5197 (2005)); neurogenesis in the adult hippocampus (e.g., Bonaguidi et al., J. Neurosci., 28:9194-9204 (2008)), cerebellum (e.g., Barneda-Zahonero et al., Mol. Biol. Cell, 20:5051-5063 (2009)), hypothalamus (e.g., Ohyama et al., Development, 135:3325-3331 (2008)), the sympathetic nervous system (e.g., Schneider et al., Neuron, 24:861-870 (1999)), and peripheral nerves.

Recent reports describe the presence of BMP7 and its receptors in various adult brain regions, including the hippocampus, pituitary and hypothalamus (Charytoniuk et al., supra (2000); Ohyama et al., Development, 132:5185-5197 (2005); Ericson et al., Development, 125:1005-1015 (1998); and Huang et al., Endocrinology, 142:2275-2283 (2001)).

As disclosed herein, BMP expression in the hypothalamus is influenced by food intake, specifically, by high fat diet. Moreover, BMP (e.g., BMP7) expression is increased upon intake of a high fat diet. Also disclosed herein are data showing that BMP7 can be used to suppress appetite, which can promote weight loss, for example, due to decreased food intake. Accordingly, the present disclosure provides compositions and methods for using BMP or BMP agents disclosed herein to modulate (e.g., suppress) appetite.

### Compositions and Methods for Modulating BMP

As disclosed herein, appetite can be modulated (e.g., suppressed) using BMP (e.g., by administering BMP to a subject). More specifically, appetite can be modulated by increasing BMP (e.g., BMP7) expression and/or by increasing the activity of a BMP receptor (e.g., a BMP7 receptor), and/or by increasing BMP signaling in the brain (e.g., hypothalamus). Such increases can include a change in expression, activity, and/or signaling, such that the resulting expression, activity, and/or signaling is greater than the expression, activity, and/or signaling prior to treatment.

In some instances, an increase in BMP expression, activity, and/or signaling can be promoted by: increasing the expression or activity of endogenous BMP or BMP receptors by administering pharmaceuticals or biologics that promote such a response; administering exogenous BMP or BMP receptors; or both. Such compositions are referred to collectively herein as BMP agents. BMP agents can include, but are not limited to, e.g., one or more of: (a) BMP2, -4, -5, -6, and/or -7 polypeptides or functional fragments or variants thereof (e.g., active (e.g., BMPR-I and/or BMPR-II activating) BMP2, -4, -5, -6, and/or -7 polypeptides or a functional fragments or analogs thereof (e.g., a mature BMP2, -4, -5, -6, and/or -7 polypeptide, e.g., a mature BMP2, -4, -5, -6, and/or -7 polypeptide described herein)); (b) peptides or protein agonists of a BMP2, -4, -5, -6, and/or -7 receptor, e.g., that increases the activity of a BMP receptor (e.g., BMPR-I and/or BMPR-II) either in the absence of BMP or by increasing or stabilizing binding of BMP2, -4, -5, -6, and/or -7 to its receptor); (c) small molecules or protein mimetics that mimic BMP2, -4, -5, -6, and/or -7 signaling activity (e.g., BMPR-I and/or BMPR-II binding activity, or SMAD phosphorylating activity); (d) small molecules that increase expression of one or more of BMP2, -4, -5, -6, and/or -7 (e.g., by binding to the promoter region of a BMP2, -4, -5, -6, and/or -7 gene); (e) antibodies or antibody fragments (e.g., a recombinant antibody or antibody fragment), e.g., antibodies or antibody fragments that bind (e.g., bind specifically) to a BMP receptor or antibodies and/or antibody fragments that stabilize or assist the binding of BMP2, -4, -5, -6, and/or -7 to a BMP2, -4, -5, -6, and/or -7 binding partner (e.g., a BMP2, -4, -5, -6, and/or -7 receptor); and that promotes biological activity of the receptor. Such antibodies or antibody fragments include, but are not limited to, for example, murine, chimeric, humanized, human, monoclonal, and polyclonal antibodies; antigen binding fragments of murine, chimeric, humanized, human, monoclonal, and polyclonal antibodies (e.g., monovalent fragments (e.g., Fab and ScFv) and engineered variants (e.g., diabodies, triabodies, minibodies, and single domain antibodies); and binding peptides containing an antigen binding portion of an BMP receptor-binding antibody (e.g., a complementarity domain region (CDR) (e.g., CDR3, CDR2, and/or CDR1); and/or (f) nucleic acids encoding a BMP2, -4, -5, -6, and/or -7 polypeptide or functional fragment or analog thereof. Such nucleic acids include genomic and cDNA sequences. BMP agents can also include BMP (polypeptides and nucleic acids) and functional equivalents of BMP (e.g., agents that target (e.g., specifically target) a selected BMP receptor (e.g., a BMP7 receptor) and evoke the same (e.g., substantially the same) biological effect produced upon binding of BMP to the receptor), such as, but not limited to, for example, pharmaceutical and biologics (including biosimilars and/or bioequivalents), such as, mimetics (e.g., protein mimetics), small molecules, antibodies and antibody fragments, and BMP receptor agonists.

BMP Polypeptides and Nucleic Acids

In some embodiments, a BMP agent can include a BMP2, -4, -5, -6, and/or -7 polypeptide or a nucleic acid molecule encoding BMP2, -4, -5, -6, and/or -7.

Polypeptides

BMP polypeptides can include, e.g., recombinant BMP polypeptides and chemically synthesized BMP polypeptides. BMP polypeptides (e.g., a mature BMP polypeptide) are themselves viable therapeutic compound because BMPs are small secreted proteins that are internalized into their target cells where they exert their activity. Although the human proteins are described herein, one of skill in the art will appreciate that when another species is the intended recipient of the treated cells, homologous proteins from that species can also be used, e.g., cow, pig, sheep, or goat. Such homologous proteins can be identified, e.g., using methods known in the art, e.g., searching available databases for homologs identified in the target species, e.g., the homologene database.

BMP2

BMP2 is 396 amino acids in length, localized to chromosome 20p12 in human. The nucleotide and amino acid sequences of human BMP2 are disclosed in Wozney et al., Science 242(4885):1528-1534 (1988). BMP2 belongs to the transforming growth factor-beta (TGFβ) superfamily. Bone morphogenetic protein induces bone formation, and BMP2 is a candidate gene for the autosomal dominant disease of fibrodysplasia (myositis) ossificans progressive. Bone morphogenetic protein 2 regulates myogenesis through dosage-dependent PAX3 expression in pre-myogenic cells, and is expressed in mesoderm under SHM control through the SOX9.

The human BMP2 amino acid sequence is shown below. Amino acids 38-268 are the TGFβ propeptide domain, and 291-396 are the TGFβ family N-terminal domain. Amino acids 283-396 are the mature peptide. The sequence is set forth in Wozney et al., Science 242:1528-1534 (1988).

(SEQ ID NO: 1)
MVAGTRCLLALLLPQVLLGGAAGLVPELGRRKFAAASSGRPSSQPSD
EVLSEFELRLLSMFGLKQRPTPSRDAVVPPYMLDLYRRHSGQPGSPA
PDHRLERAASRANTVRSFHHEESLEELPETSGKTTRRFFFNLSSIPT
EEFITSAELQVFREQMQDALGNNSSFHHRINIYEIIKPATANSKFPV
TRLLDTRLVNQNASRWESFDVTPAVMRWTAQGHANHGFVVEVAHLEE
KQGVSKRHVRISRSLHQDEHSWSQIRPLLVTFGHDGKGHPLHKREKR
QAKHKQRKRLKSSCKRHPLYVDFSDVGWNDWIVAPPGYHAFYCHGEC
PFPLADHLNSTNHAIVQTLVNSVNSKIPKACCVPTELSAISMLYLDE
NEKVVLKNYQDMVVEGCGCR

The mature form of BMP2 contains four potential N-linked glycosylation sites per polypeptide chain, and four potential disulfide bridges. See UniProt entry No. P12643; HomoloGene: 926; GenBank Acc. Nos. NM_{—}001200.2 (mRNA) and NP_{—}001191.1 (protein).

In some embodiments, the BMP agent is a BMP2 polypeptide, e.g., human BMP2, e.g., a mature BMP2 polypeptide, e.g., a BMP2 polypeptide that includes amino acids 283-396 of SEQ ID NO:1.

BMP4

BMP4 induces cartilage and bone formation, and is important in mesoderm induction, tooth development, limb formation and fracture repair. The amino acid sequence of the BMP4 preproprotein is shown below. Amino acids 41-276 are the TGFβ propeptide domain, and 302-408 are the TGFβ family N-terminal domain. Amino acids 293-408 are the mature peptide. The sequence is set forth in Wozney et al., Science 242:1528-1534 (1988).

(SEQ ID NO: 2)
MIPGNRMLMVVLLCQVLLGGASHASLIPETGKKKVAEIQGHAGGRR
SGQSHELLRDFEATLLQMFGLRRRPQPSKSAVIPDYMRDLYRLQSG
EEEEEQIHSTGLEYPERPASRANTVRSFHHEEHLENIPGTSENSAF
RFLFNLSSIPENEAISSAELRLFREQVDQGPDWERGFHRINIYEVM
KPPAEVVPGHLITRLLDTRLVHHNVTRWETFDVSPAVLRWTREKQP
NYGLAIEVTHLHQTRTHQGQHVRISRSLPQGSGNWAQLRPLLVTFG
HDGRGHALTRRRRAKRSPKHHSQRARKKNKNCRRHSLYVDFSDVGW
NDWIVAPPGYQAFYCHGDCPFPLADHLNSTNHAIVQTLVNSVNSSI
PKACCVPTELSAISMLYLDEYDKVVLKNYQEMVVEGCGCR

The mature form of BMP4 contains four potential N-linked glycosylation sites per polypeptide chain. A variant exists in which V152 is an A. See UniProt Accession No. P12644; HomoloGene: 7247; GenBank Acc. Nos. NM_{—}001202.3 (mRNA, var. 1) and NP_{—}001193.2 (protein, var. 1); NM_{—}130850.2 (mRNA, var. 2) and NP_{—}570911.2 (protein, var. 2). and NM_{—}130851.2 (mRNA, var. 3) and NP_{—}570912.2 (protein, var. 3).

In some embodiments, the BMP agent is a BMP4 polypeptide, e.g., human BMP4, e.g., a mature BMP4 polypeptide, e.g., a BMP4 polypeptide that includes amino acids 293-408 of SEQ ID NO:2.

BMP5

The BMP5 preproprotein is a 454 amino acid protein, as shown below. BMP5 induces cartilage and bone formation. The amino acid sequence of BMP5 is set forth in Celeste et al., Proc. Natl. Acad. Sci. U.S.A., 87, 9843-9847, 1990.

(SEQ ID NO: 3)
MHLTVFLLKGIVGFLWSCWVLVGYAKGGLGDNHVHSSFIYRRLRNH
ERREIQREILSILGLPHRPRPFSPGKQASSAPLFMLDLYNAMTNEE
NPEESEYSVRASLAEETRGARKGYPASPNGYPRRIQLSRTTPLTTQ
SPPLASLHDTNFLNDADMVMSFVNLVERDKDFSHQRRHYKEFRFDL
TQIPHGEAVTAAEFRIYKDRSNNRFENETIKISIYQIIKEYTNRDA
DLFLLDTRKAQALDVGWLVFDITVTSNHWVINPQNNLGLQLCAETG
DGRSINVKSAGLVGRQGPQSKQPFMVAFFKASEVLLRSVRAANKRK
NQNRNKSSSHQDSSRMSSVGDYNTSEQKQACKKHELYVSFRDLGWQ
DWIIAPEGYAAFYCDGECSFPLNAHMNATNHAIVQTLVHLMFPDHV
PKPCCAPTKLNAISVLYFDDSSNVILKKYRNMVVRSCGCH

The mature BMP5 protein is believed to be amino acids 323-454 of SEQ ID NO:3, and has four potential N-linked glycosylation sites per polypeptide chain, and four potential disulfide bridges. See UniProt Accession Nos. P22003; Q9H547; or Q9NTM5; HomoloGene: 22412; GenBank Acc. Nos. NM_{—}021073.2 (mRNA) and NP_{—}066551.1 (protein).

In some embodiments, the BMP agent is a BMP5 polypeptide, e.g., human BMP5, e.g., a mature BMP5 polypeptide, e.g., a BMP4 polypeptide that includes amino acids 323-454 of SEQ ID NO:3.

BMP6

BMP6 is an autocrine stimulator of chondrocyte differentiation, and is involved in the development of embryonic neural, and urinary systems, as well as growth and differentiation of liver and keratinocytes. BMP6 knockout mice are viable and show a slight delay in ossification of the sternum. BMP6 (precursor) is a 57 kD protein, 513 amino acids in length, localized to chromosome 6p24 in human. The nucleotide and amino acid sequence of human BMP6 is disclosed in U.S. Pat. No. 5,187,076. BMP6 is predicted to be synthesized as a precursor molecule which is cleaved to yield a 132 amino acid mature polypeptide with a calculated molecular weight of approximately 15 Kd. The mature form of BMP6 contains three potential N-linked glycosylation sites per polypeptide chain. The active BMP6 protein molecule is likely a dimer. Processing of BMP6 into the mature form involves dimerization and removal of the N-terminal region in a manner analogous to the processing of the related protein TGFβ (Gentry et al., Molec. Cell. Biol. 8:4162 (1988); Dernyck et al., Nature 316:701 (1985)). The human BMP6 precursor is shown below. The mature polypeptide is believed to include amino acids 374-513 of SEQ ID NO:4. Other active BMP6 polypeptides include polypeptides including amino acids 382-513, 388-513 and 412-513 of SEQ ID NO:4.

(SEQ ID NO: 4)
MPGLGRRAQWLCWWWGLLCSCCGPPPLRPPLPAAAAAAAGGQLLGD
GGSPGRTEQPPPSPQSSSGFLYRRLKTQEKREMQKEILSVLGLPHR
PRPLHGLQQPQPPALRQQEEQQQQQQLPRGEPPPGRLKSAPLFMLD
LYNALSADNDEDGASEGERQQSWPHEAASSSQRRQPPPGAAHPLNR
KSLLAPGSGSGGASPLTSAQDSAFLNDADMVMSFVNLVEYDKEFSP
RQRHHKEFKFNLSQIPEGEVVTAAEFRIYKDCVMGSFKNQTFLISI
YQVLQEHQHRDSDLFLLDTRVVWASEEGWLEFDITATSNLWVVTPQ
HNMGLQLSVVTRDGVHVHPRAAGLVGRDGPYDKQPFMVAFFKVSEV
HVRTTRSASSRRRQQSRNRSTQSQDVARVSSASDYNSSELKTACRK
HELYVSFQDLGWQDWIIAPKGYAANYCDGECSFPLNAHMNATNHAI
VQTLVHLMNPEYVPKPCCAPTKLNAISVLYFDDNSNVILKKYRNMV
VRACGCH

The human BMP6 promoter has been characterized (See Tamada et al., Biochim Biophys Acta. 1998, 1395(3):247-51), and can be used in methods described herein. See UniProt Accession No. P22004; HomoloGene: 1300; GenBank Acc. Nos. NM_{—}001718.4 (mRNA) and NP_{—}001709.1 (protein).

Administration, antisense treatment, and quantitation of BMP6 are described in Boden et al. (Endocrinology Vol. 138, No. 7 2820-2828).

In some embodiments, the BMP agent is a BMP6 polypeptide, e.g., human BMP6, e.g., a mature BMP6 polypeptide, e.g., a BMP6 polypeptide that includes amino acids 374-513 of SEQ ID NO:4, amino acids 382-513 of SEQ ID NO:4, amino acids 388-513 of SEQ ID NO:4, or amino acids 412-513 of SEQ ID NO:4.

BMP7

BMP7 also belongs to the TGFβ superfamily. It induces cartilage and bone formation, and may be the osteoinductive factor responsible for the phenomenon of epithelial osteogenesis. BMP7 plays a role in calcium regulation and bone homeostasis, and in the regulation of anti-inflammatory response in the adult gut tissue. The sequence of BMP7 is shown below:

(SEQ ID NO: 5)
MHVRSLRAAAPHSFVALWAPLFLLRSALADFSLDNEVHSSFIHRR
LRSQERREMQREILSILGLPHRPRPHLQGKHNSAPMFMLDLYNAM
AVEEGGGPGGQGFSYPYKAVFSTQGPPLASLQDSHFLTDADMVMS
FVNLVEHDKEFFHPRYHHREFRFDLSKIPEGEAVTAAEFRIYKDY
IRERFDNETFRISVYQVLQEHLGRESDLFLLDSRTLWASEEGWLV
FDITATSNHWVVNPRHNLGLQLSVETLDGQSINPKLAGLIGRHGP
QNKQPFMVAFFKATEVHFRSIRSTGSKQRSQNRSKTPKNQEALRM
ANVAENSSSDQRQACKKHELYVSFRDLGWQDWIIAPEGYAAYYCE
GECAFPLNSYMNATNHAIVQTLVHFINPETVPKPCCAPTQLNAIS
VLYFDDSSNVILKKYRNMVVRACGCH

Amino acids 1-29 are a potential signal sequence; 30-431 are the prepropeptide, and 293-431 are the mature protein. The mature form of BMP7 contains four potential N-linked glycosylation sites per polypeptide chain, and four potential disulfide bridges. See UniProt Accession No. P18075; HomoloGene: 20410; GenBank Acc. Nos. NM_{—}001719.2 (mRNA) and NP_{—}001710.1 (protein).

In some embodiments, the BMP agent is a BMP7 polypeptide, e.g., human BMP7, e.g., a mature BMP7 polypeptide, e.g., a BMP7 polypeptide that includes amino acids 293-431 of SEQ ID NO:5.

In some embodiments, BMP polypeptides can be at least 50%, 60%, 70%, 80%, 85%, 90%, 95%, 98%, 99%, or 100% homologous to a BMP sequence known in the art or described herein, e.g., SEQ ID NO:1, 2, 3, 4, and/or 5.

To determine the percent identity of two amino acid sequences, or of two nucleic acid sequences, such as those nucleic acid sequences disclosed below, the sequences are aligned for optimal comparison purposes (e.g., gaps can be introduced in one or both of a first and a second amino acid or nucleic acid sequence for optimal alignment and non-homologous sequences can be disregarded for comparison purposes). In general, the length of a reference sequence aligned for comparison purposes is 100% of the length of the reference sequence (e.g., the full length). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The determination of percent identity between two amino acid sequences could be accomplished using the BLAST 2.0 program. Sequence comparison is performed using an ungapped alignment and using the default parameters (Blossom 62 matrix, gap existence cost of 11, per residue gapped cost of 1, and a lambda ratio of 0.85). The mathematical algorithm used in BLAST programs is described in Altschul et al. (Nucleic Acids Res. 25:3389-3402, 1997). Useful BMP encoding polypeptide sequences or polypeptide fragments can have up to about 20 (e.g., up to about 10, 5, or 3) amino acid deletions, additions, or substitutions, such as conservative substitutions, to be useful for the compositions and methods described herein. Conservative amino acid substitutions are known in the art.

In some embodiments, a BMP polypeptide can be modified, e.g., to increase one or more of the activity, stability, binding activity, and/or binding specificity of the polypeptide. For example, modifications can be made to a polypeptide that result in pharmacokinetic properties of the protein which are desirable for use in protein therapy. For example, such modifications can result in an increase in cellular uptake, circulatory half-life, rate of clearance and reduced immunogenicity. Several art-recognized approaches are known that are useful to optimize the therapeutic activity of a protein compound, e.g., a compound described herein such as a BMP2, -4, -5, -6, and/or -7 polypeptide.

For recombinant proteins, the choice of expression system can influence pharmacokinetic characteristics. Differences between expression systems in post-translational processing can lead to recombinant proteins of varying molecular size and charge, which can affect, for example, cellular uptake, circulatory half-life, rate of clearance and immunogenicity. The pharmacokinetic properties of the protein may be optimized by the appropriate selection of an expression system, such as selection of a bacterial, viral, or mammalian expression system. Exemplary mammalian cell lines useful in expression systems for therapeutic proteins are Chinese hamster ovary, (CHO) cells, the monkey COS-1 cell line and the CV-1 cell line.

A protein can be chemically altered to enhance the pharmacokinetic properties while maintaining activity. The protein can be covalently linked to a variety of moieties, altering the molecular size and charge of the protein and consequently its pharmacokinetic characteristics. The moieties are preferably non-toxic and biocompatible. In some embodiments, polyethylene glycol (PEG) can be covalently attached to the protein (PEGylation). See, e.g., Poly(ethylene glycol): Chemistry and Biological Applications, Harris and Zalipsky, eds., ACS Symposium Series, No. 680, 1997; Harris et al., Clinical Pharmacokinetics 40:7, 485-563 (2001)). In another embodiment, the protein can be similarly linked to oxidized dextrans via an amino group. (See Sheffield, Current Drug Targets—Cardiovas. and Haemat. Dis. 1:1, 1-22 (2001)).

Furthermore, the protein compounds can be chemically linked to another protein. The protein can be cross-linked carrier protein to form a larger molecular weight complex with improved cellular uptake. In some embodiments, the carrier protein can be a serum protein, such as albumin. The protein can be attached to one or more albumin molecules via a bifunctional cross-linking recompound. The cross-linking recompound may be homo- or heterofunctional. In another embodiment, the protein can cross-link with itself to form a homodimer, trimer, or higher analog. Again, either heterobifunctional or homobifunctional cross-linking recompounds can be used to form the dimers or trimers. (See Stykowski et al., Proc. Natl. Acad. Sci. USA, 95, 1184-1188 (1998)).

Nucleic Acids

In some embodiments, the BMP agent can be a BMP nucleic acid that enhances BMP expression, activity, and/or signaling as described herein, which can include, e.g., a BMP nucleic acid, e.g., a BMP2, -4, -5, -6, and/or -7 encoding nucleic acid sequence or a biologically active fragment or analog thereof (e.g., a nucleic acid encoding one or more of SEQ ID NOs:1-5 or a nucleic acid encoding a polypeptide with a sequence with at least 50%, 60%, 70%, 80%, 85%, 90%, 95%, 98%, 99%, or 100% homology to one or more of SEQ ID NOs:1-5), and any of: a promoter sequence, e.g., a promoter sequence from a BMP2, -4, -5, -6, and/or -7 gene or from another gene; an enhancer sequence, e.g., 5′ untranslated region (UTR), e.g., a 5′ UTR from a BMP2, -4, -5, -6, and/or -7 gene or from another gene, a 3′ UTR, e.g., a 3′ UTR from a BMP2, -4, -5, -6, and/or -7 gene or from another gene; a polyadenylation site; an insulator sequence; or another sequence that enhances the expression of BMP2, -4, -5, -6, and/or -7.

In another embodiment, the level of BMP2, -4, -5, -6, and/or -7 protein is increased by increasing the level of expression of an endogenous BMP2, -4, -5, -6, and/or -7 gene, e.g., by increasing transcription of the BMP2, -4, -5, -6, and/or -7 gene or increasing BMP2, -4, -5, -6, and/or -7 mRNA stability. In some embodiments, transcription of the BMP2, -4, -5, -6, and/or -7 gene is increased by: altering the regulatory sequence of the endogenous BMP2, -4, -5, -6, and/or -7 gene, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the BMP2, -4, -5, -6, and/or -7 gene to be transcribed more efficiently. In some embodiments, the nucleic acid encodes or increases transcription of BMP7.

The nucleic acids described herein, e.g., a nucleic acid encoding a BMP2, -4, -5, -6, and/or -7 polypeptide as described herein, can be incorporated into a gene construct. The methods described herein can use such expression vectors for in vitro transfection and expression of a BMP2, -4, -5, -6, and/or -7 polypeptide described herein in particular cell types, e.g., stem cells, e.g., pluripotent mesenchymal stem cells. Expression constructs of such components can be administered in any biologically effective carrier, e.g., any formulation or composition capable of effectively delivering the component gene to cells in vivo. Approaches include insertion of a subject gene in viral vectors including recombinant retroviruses, adenovirus, adeno-associated virus, and herpes simplex virus-1, or recombinant bacterial or eukaryotic plasmids.

Viral vectors transfect cells directly, and infection of cells with a viral vector generally has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid. Retroviral vectors, adenovirus-derived vectors, and adeno-associated virus vectors can also be used as a recombinant gene delivery system for the transfer of exogenous genes. These vectors provide efficient delivery of genes into cells, and the transferred nucleic acids are generally stably integrated into the chromosomal DNA of the host. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14, and other standard laboratory manuals.

Non-viral methods can also be employed to cause expression of an nucleic acid compound described herein (e.g., a BMP2, -4, -5, -6, and/or -7 polypeptide encoding nucleic acid) into a cell. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of the subject gene by the targeted cell. Plasmid DNA can be delivered with the help of, for example, cationic liposomes (e.g., LIPOFECTIN™) or derivatized (e.g., antibody conjugated), polylysine conjugates, gramicidin S, artificial viral envelopes or other such intracellular carriers, as well as direct injection of the gene construct or CaPO₄ precipitation. Other embodiments include plasmid injection systems such as are described in Meuli et al., J. Invest. Dermatol. 116(1):131-135 (2001); Cohen et al., Gene Ther 7(22):1896-905 (2000); or Tam et al., Gene Ther. 7(21):1867-74 (2000).

The nucleic acids described herein can be incorporated into a gene construct that facilitates administration and uptake of the nucleic acid by a selected cell or tissue type. The invention features expression vectors for in vivo transfection and expression of a BMP2, -4, -5, -6, and/or -7 polypeptide described herein in particular cell types. Expression constructs of such components may be administered in any biologically effective carrier, e.g. any formulation or composition capable of effectively delivering the component gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (e.g., LIPOFECTIN™) or derivatized (e.g. antibody conjugated), polylysine conjugates, gramicidin S, artificial viral envelopes or other such intracellular carriers, as well as direct injection of the gene construct or CaPO₄ precipitation carried out in vivo.

One approach for in vivo introduction of nucleic acid into a cell is by use of a viral vector containing nucleic acid, e.g. a cDNA, encoding an alternative pathway component described herein. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271-78 (1990)). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel et al. (eds.) Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include *Crip, *Cre, *2 and *Am. Retroviruses have been used to introduce a variety of genes into many different cell types, including epithelial cells, in vitro and/or in vivo (see for example Eglitis, et al., Science 230:1395-1398 (1985); Danos and Mulligan, Proc. Natl. Acad. Sci. USA 85:6460-6464 (1988); Wilson et al., Proc. Natl. Acad. Sci. USA 85:3014-3018 (1988); Armentano et al., Proc. Natl. Acad. Sci. USA 87:6141-6145 (1990); Huber et al., Proc. Natl. Acad. Sci. USA 88:8039-8043 (1991); Ferry et al., Proc. Natl. Acad. Sci. USA 88:8377-8381 (1991); Chowdhury et al., Science 254:1802-1805 (1991); van Beusechem et al., Proc. Natl. Acad. Sci. USA 89:7640-7644 (1992); Kay et al., Human Gene Therapy 3:641-647 (1992); Dai et al., Proc. Natl. Acad. Sci. USA 89:10892-10895 (1992); Hwu et al., J. Immunol. 150:4104-4115 (1993); U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 d1324 or other strains of adenovirus (e.g., Ad2, Ad3, Ad7 etc.) are known to those skilled in the art. Recombinant adenoviruses can be advantageous in certain circumstances in that they are not capable of infecting non-dividing cells and can be used to infect a wide variety of cell types, including epithelial cells (Rosenfeld et al. (1992), supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. (1998), supra; Haj-Ahmand and Graham, J. Virol. 57:267 (1986)).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992)). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989)). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993)).

In addition to viral transfer methods, such as those illustrated above, non-viral methods can also be employed to cause expression of an nucleic acid agent described herein (e.g., a BMP2, -4, -5, -6, and/or -7 polypeptide encoding nucleic acid) in the tissue of a subject. Most nonviral methods of gene transfer rely on normal mechanisms used by mammalian cells for the uptake and intracellular transport of macromolecules. In some embodiments, non-viral gene delivery systems of the present invention rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther 7(22):1896-905 (2000); or Tam et al., Gene Ther. 7(21):1867-74 (2000).

In a representative embodiment, a gene encoding an alternative pathway component described herein can be entrapped in liposomes bearing positive charges on their surface (e.g., lipofectins) and (optionally) which are tagged with antibodies against cell surface antigens of the target tissue (Mizuno et al., No Shinkei Geka 20:547-551 (1992); PCT publication WO91/06309; Japanese patent application 1047381; and European patent publication EP-A-43075).

In clinical settings, the gene delivery systems for the therapeutic gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., PNAS 91: 3054-3057 (1994)).

In some embodiments, the BMP nucleic acid can be engineered to target neurons or optimally express either mRNA and/or the protein encoded thereby in the brain (e.g., in the hypothalamus).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

### Peptide Mimetics

In some embodiments, the BMP agent is a peptide mimetic (e.g., either a peptide or nonpeptide peptide mimetic). Synthesis of nonpeptide compounds that mimic peptide sequences is known in the art. Nonpeptide compounds that mimic, for example, BMP7, are specifically contemplated by the present invention. Such peptide mimetics include BMP peptides that can be modified according to methods known in the art for producing peptidomimetics. See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem., 278:45746, 2003. In some cases, these modified peptidomimetic versions of the peptides and fragments disclosed herein exhibit enhanced stability in vivo, relative to the non-peptidomimetic peptides. Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences. Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as α-amino phosphonic acids and α-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules. These sequences can be modified, e.g., by biotinylation of the amino terminus and amidation of the carboxy terminus.

### Additional BMP Agents

In some embodiments, a BMP agent can include modulators of TDF-like receptors, e.g., as disclosed in U.S. Pat. No. 7,482,329, and U.S. Publication No. 2010/0015150 (e.g., compounds termed “Thrasos compounds that bind to ALK3”), which is incorporated by reference herein in its entirety. In some embodiments, a BMP agent can include peptides with the amino acid sequences shown below:

| | |
|---|---|
| CIVNSSDDFLCKKYRS | (SEQ ID NO: 6) |
| CYFNDSSQVLCKRYRS | (SEQ ID NO: 7) |

or peptide mimetics thereof, e.g., as described herein.

In some embodiments, a BMP agent can include the BMP7 mimetic AA123 (Thrasos Inc., Hopkinton, Ma). Similar to BMP7, AA123 binds specifically to immobilized extracellular domains (ECD) of ALK3 and BMPR-II and promotes SMAD01 translocation. Unlike BMP7, AA123 does not bind to ALK6.

In some embodiments, a BMP agent can include BMP2 mimetics and other proteins that increase or enhance BMP-2 activity such as, e.g., LIM mineralization protein-1 (LMP-1) and/or SVAK-3 as disclosed by Okada et al. (Cell. Biochem. Funct., 27:526-534 (2009)); and B2A2, as disclosed by Lin et al. (Journal of Bone and Mineral Res., 20:693-703 (2005)).

Any of the peptides described herein, including the variant forms described herein, can further include a heterologous polypeptide. The heterologous polypeptide can be a polypeptide that increases the circulating half-life of the peptide to which it is attached (e.g., fused, as in a fusion protein). The heterologous polypeptide can be an albumin (e.g., a human serum albumin or a portion thereof) or a portion of an immunoglobulin (e.g., the Fc region of an IgG). In some instances, the heterologous peptide can be a polypeptide that increases trafficking of the peptide to which it is attached across the blood brain barrier. Alternatively or in addition, the heterologous peptide can be a polypeptide that targets the peptide to which it is attached to the brain (e.g., to the hypothalamus). Such methods are described in the art (see, e.g., Begley, J. Pharm. Pharmacol., 48:136-146 (1996)).

The present disclosure also contemplates synthetic mimicking compounds. As is known in the art, peptides can be synthesized by linking an amino group to a carboxyl group that has been activated by reaction with a coupling agent, such as dicyclohexylcarbodiimide (DCC). The attack of a free amino group on the activated carboxyl leads to the formation of a peptide bond and the release of dicyclohexylurea. It can be necessary to protect potentially reactive groups other than the amino and carboxyl groups intended to react. For example, the (α-amino group of the component containing the activated carboxyl group can be blocked with a tertbutyloxycarbonyl group. This protecting group can be subsequently removed by exposing the peptide to dilute acid, which leaves peptide bonds intact. With this method, peptides can be readily synthesized by a solid phase method by adding amino acids stepwise to a growing peptide chain that is linked to an insoluble matrix, such as polystyrene beads. The carboxyl-terminal amino acid (with an amino protecting group) of the desired peptide sequence is first anchored to the polystyrene beads. The protecting group of the amino acid is then removed. The next amino acid (with the protecting group) is added with the coupling agent. This is followed by a washing cycle. The cycle is repeated as necessary.

In some embodiments, the mimetics of the present disclosure are peptides having sequence homology to a herein-described BMP peptide. These mimetics include, but are not limited to, peptides in which L-amino acids are replaced by their D-isomers. One common methodology for evaluating sequence homology, and more importantly statistically significant similarities, is to use a Monte Carlo analysis using an algorithm written by Lipman and Pearson to obtain a Z value. According to this analysis, a Z value greater than 6 indicates probable significance, and a Z value greater than 10 is considered to be statistically significant (Pearson and Lipman, Proc. Natl. Acad. Sci. (USA), 85:2444-2448, 1988; Lipman and Pearson, Science, 227:1435-1441, 1985. More generally, the peptide ligands described herein and the mimetics described above can be synthesized using any known methods, including tea-bag methodology or solid phase peptide synthesis procedures described by Merrifield et al., Biochemistry, 21:5020-5031, 1982; Houghten Wellings, Proc. Natl. Acad. Sci. (USA), 82:5131-5135, 1985; Atherton, Methods in Enzymology, 289:44-66, 1997, or Guy and Fields, Methods in Enzymology, 289:67-83, 1997, or using a commercially available automated synthesizer.

### Pharmaceutical Formulations

The BMP agent can be incorporated into pharmaceutical compositions suitable for administration to a subject, e.g., a human. In some instances, the pharmaceutical formulation can be optimized for systemic administration and/or use in the central nervous system (e.g., the brain). Such compositions typically include one or more of the BMP agents disclosed herein and a pharmaceutically acceptable carrier. A pharmaceutically acceptable carrier can include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions. Such supplementary active compounds can include, but are not limited to, e.g., peroxisome proliferator-activated receptor gamma (PPAR), Retinoid X receptor, alpha (RxR), insulin, T3, a thiazolidinedione (TZD), retinoic acid, another BMP (e.g., the BMP being used and one or more of BMP 1, 2, 3, 4, 5, 6, or 7), vitamin A, retinoic acid, insulin, glucocorticoid or agonist thereof; Wingless-type (Wnt), e.g., Wnt-1, Insulin-like Growth Factor-1 (IGF-1), or other growth factor, e.g., Epidermal growth factor (EGF), Fibroblast growth factor (FGF), Transforming growth factor (TGF)-β, TGF-γ, Tumor necrosis factor alpha (TNFα), Macrophage colony stimulating factor (MCSF), Vascular endothelial growth factor (VEGF) and/or Platelet-derived growth factor (PDGF).

A pharmaceutical composition can be formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an agent described herein) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL™ (sodium carboxymethyl starch), or corn starch; a lubricant such as magnesium stearate or STEROTES™; a glidant such as colloidal silicon dioxide; a sweetening agent such as sucrose or saccharin; or a flavoring agent such as peppermint, methyl salicylate, or orange flavoring.

For transmucosal or transdermal administration, penetrants appropriate to the barrier to be permeated can be included. Such penetrants are generally known, and include, for example, for transmucosal administration, detergents, bile salts, and fusidic acid derivatives. In addition, transdermal compositions can be formulated into ointments, salves, gels, or creams as generally known in the art

In some embodiments, the BMP agent can be encapsulated or can be contained in a matrix or carrier. The agent can be provided in a matrix capable of delivering the agent to the chosen site.

Matrices can provide slow release of the agent and provide proper presentation and appropriate environment for cellular infiltration. Matrices can be formed of materials presently in use for other implanted medical applications. The choice of matrix material is based on any one or more of: biocompatibility, biodegradability, mechanical properties, cosmetic appearance and interface properties. One example is a collagen matrix. Carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, the pharmaceutical composition can be included in a container, pack, or dispenser together with instructions for administration, e.g., in a kit.

### Routes of Administration

As disclosed herein, BMP administered locally, to the brain by intracerebroventricular (ICV) administration, and systemically suppress appetite. Also as disclosed herein, BMP crosses the blood brain barrier. Accordingly, any BMP agent capable of crossing the blood brain barrier (e.g., BMP7) can be administered either systemically or locally. Exemplary systemic routes of administration include, but are not limited to, parenteral, intravenous, intramuscular, intraperitoneal, intradermal, subcutaneous, percutaneous injection, oral, transdermal, and transmucosal administration. In some instances, the BMP agent can be administered to a site of adipose tissue, e.g., a subcutaneous or omentum adipose pad. Exemplary local routes of administration include, but are not limited to, ICV administration.

In some embodiments, BMP agents can be administered using a catheter and a pump. For example, BMP agents can be present in a refillable reservoir and can be propelled through the catheter by the pump. In some instances, the catheter can direct the BMP agents systemically, e.g., intravenously. Alternatively, the catheter can direct the BMP agents locally to the brain. The catheter and pump can be installed for long-term or short-term use.

Alternatively or in addition, the BMP agents may be administered according to any of the Food and Drug Administration approved methods, for example, as described in CDER Data Standards Manual, version number 004 (which is available at fda.give/cder/dsm/DRG/drg00301.htm).

### Effective Dose

Toxicity and therapeutic efficacy of the compositions described herein can be determined by standard pharmaceutical procedures, using either cells in culture or experimental animals to determine the LD₅₀ (the dose lethal to 50% of the population) and the ED₅₀ (the dose therapeutically effective in 50% of the population). The dose ratio between toxic and therapeutic effects is the therapeutic index and can be expressed as the ratio LD₅₀/ED₅₀. Compounds that exhibit large therapeutic indices are preferred.

### Methods of Treatment

The present disclosure provides methods for modulating (e.g., suppressing) appetite using BMP. In some embodiments, these methods can include selecting a subject for treatment (e.g., a subject in need of decreased appetite), and administering to the subject one or more BMP agents under conditions and in amounts sufficient to promote a decrease in appetite in the subject. The methods can optionally further include: repeating administration of the one or more BMP agents, for example, for a period sufficient to yield a decrease in body weight of the subject (weight can be assessed by weighing the subject), for a period prescribed by a health care professional, for a period elected by the subject, or for a period sufficient to reduce the subject's BMI; and/or monitoring food intake by the subject, each of which can be performed before and after administration of the one or more BMPs and/or before and after the subject has completed treatment (e.g., once no further administrations of one or more BMP agents are required). In some embodiments, treatment can be continued until a subject's BMI is below 30. In some embodiments, treatment can be continued until a subject's BMI is below 25.

In some embodiments, the methods include administering the compound in combination with a second treatment, e.g., a second treatment for obesity or an obesity-related disorder, e.g., type II diabetes. For example, the second treatment can be insulin, orlistat, phendimetrazine, and/or phentermine.

### Subject Selection

In some embodiments, the methods include selecting a subject for treatment. Subject selection can include evaluating a subject for one or more of: weight, adipose tissue stores, adipose tissue morphology, insulin levels, insulin metabolism, glucose levels, thermogenic capacity, and cold sensitivity. The evaluation can be performed before, during, and/or after the administration of the compound. For example, the evaluation can be performed at least 1 day, 2 days, 4, 7, 14, 21, 30 or more days before and/or after the administration.

In some embodiments, a subject can include a human or non-human animal that would benefit therapeutically from treatment with the compositions and methods disclosed herein. Such subjects can include, for example, obese subjects (e.g., obese subjects, as defined herein, that have been clinically diagnosed, and/or obese subjects, as defined herein, without clinical diagnosis); subjects at risk for obesity, such as overweight subjects (e.g., overweight subjects, as defined herein, that have been clinically diagnosed, and/or overweight subjects, as defined herein, without clinical diagnosis); subjects with a tendency to overeat, subjects with an eating disorder, compulsive overeaters, and/or subjects with food addiction (e.g., regardless of BMI); subjects with glucose intolerance; and/or subjects that choose to decrease their appetite.

As used herein, “obesity” refers to a disorder in a subject, wherein the subject's weight exceeds their ideal weight, according to standard tables, by 20% or more, e.g., 25%, 30%, 40%, and 50%, or more. Obese can also mean an individual with a body mass index (BMI) of 30 or more, e.g., 30-35 and 35-40 or more. For example, a subject diagnosed with class I obesity has a BMI range of 30-34.9. A subject with class II obesity has a BMI range of 35.0-39.9. A subject with class III obesity has a BMI greater than 40. “Overweight” refers to a subject with a BMI range of 25.0-29.9.

### EXAMPLES

The invention is further described in the following examples, which do not limit the scope of the invention described in the claims.

### Example 1

### BMP Expression in the Hypothalamus

The experiments presented below were performed to characterize the expression profile of BMP7 in the hypothalamus under conditions of varying nutrient availability.

Neurological expression of BMP7 was assessed using LacZ reporter staining, in situ hybridization, and confoccal fluorescent microscopy.

LacZ Reporter Staining

Brain tissue from animals in which beta-galactosidase is inserted into the BMP7 locus was assessed as follows. Briefly, whole brains were fixed in 4% buffered formaldehyde for 2-6 hours at 4° C. Fixed brains were then prepared for frozen sectioning in 10%, 20%, and 30% sucrose, for 8 h at 4° C., before embedding in OCT. Sections of 8 um thickness were prepared and post fixed for 5 min in ice-cold 4% buffered formaldehyde. Tissues were rinsed (PBS, 0.02% NP-40, 0.01 sodium deoxycholate, 2 mM MgCl2) and stained (1 mg/ml X-Gal in rinse buffer) at 37° C. overnight in the dark. Sections were repeatedly washed in PBS, counterstained with nuclear fast red, and dehydrated through ethanol/xylene series before mounting.

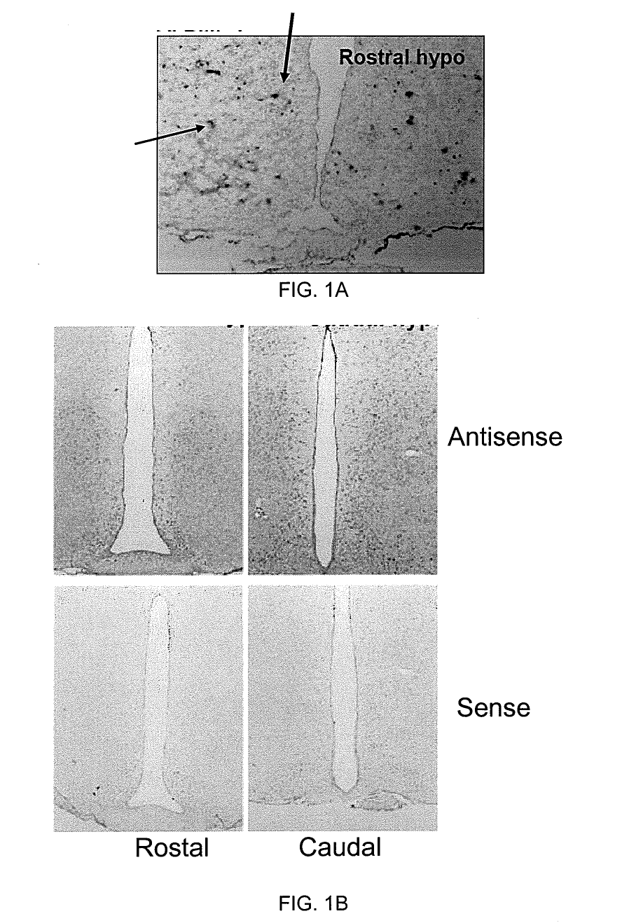

As shown in FIG. 1A, BMP7 is localized to the hypothalamus (around the third ventricle) in coronal sections. BMP7 expression was observed in both rostral and caudal sections.

In Situ Hybridization

Brain tissue from streptozotocin treated C57BL/6 mice were fixed by perfusing 4% PFA through the mice. Tissue was further fixed overnight at room temperature in 10% formalin, dehydrated through a series of ethanol washes and processed for paraffin embedding. Paraffin sections were mounted on Superfrost Plus Slides (Fisher Scientific) and used for in situ hybridization. Riboprobes were in vitro transcribed and labeled with Digitoxin using DIG RNA Labeling Mix (Roche) and the Riboprobe© Combination System (Promega). cRNA sense and antisense probes to the region corresponding to nucleotides 1037 to 1857 of Bmpr2 were generated. Slides were cleared through xylene, delipidated with chloroform, and rehydrated through a graded ethanol series. Slides were post-fixed in 4% PFA, rinsed in PBS, incubated for 10 min at 25° C. in proteinase K (20 ug/ml) in a buffer containing 50 mM Tris and 5 mM EDTA (pH 8.0). Slides were then fixed again in 4% PFA, acetylated with acetic anhydride, dehydrated and exposed to either denatured antisense or sense probes in hybridization buffer (50% formamide, 10% dextran sulfate, 1 Denhardt's solution, 300 mM NaCl, 2 mM EDTA (pH 8), 0.5 mg/ml salmon sperm DNA). Hybridization was performed at 55° C. overnight in a humidity chamber containing 4 SSC and 50% formamide. Hybridized slides were washed in 2× SSC at room temperature for 2 hrs; 50% formamide, 1 SSC at 55° C. for 2 hrs; 0.5 SSC at 37° C. for 30 min; and were exposed to 20 ug/ml RNase A in 0.5×SSC 30 min at 37° C. Slides were rinsed in PBS, and specific signal was detected with anti-Digoxigenin-AP, Fab fragments (1:500) (Roche). Slides were then washed thoroughly in PBS-T and incubated with a 100 mM Tris buffer (pH 9.5) containing chromogenic substrates for alkaline phosphatase (168.5 ul of 100 mg/ml nitro blue tetrazolium salt in dimethylformamide and 175 ul of 50 mg/ml 5-bromo-4-chloro-3-indoyl phosphate/toluidinium salt in dimethylformamide added to 50 ml of the Tris buffer). The development of a purple color is indicative of positive signal. Tissues were counterstained with nuclear fast red. Photographs were taken at 100× magnification.

As shown in FIG. 1B, purple color was specifically observed in both rostal and caudal hypothalamus sections. This observation confirms that BMP7 is expressed in the mouse brain, as shown in FIG. 1A.

Confocal Fluorescent Microscopy

Mice were anesthetized with Avertin and transcardially perfused with PBS followed by Amresco Tissue Fixative. Brains were removed from the skull and post-fixed overnight in Amresco, followed by 24 hr incubations in 5%, 20% and 30% sucrose for cryoprotection. Brains were flash-frozen in OCT and stored at −80 until sectioning. Cryostat sections at 10 uM were sliced and then dried overnight, prior to cold-Acetone fixation and staining Sections were then incubated in 200 μl of a 0.3% Sudan Black solution in 70% ethanol for 30 min at room temperature to block autofluorescence. Slides were rinsed with IHC rinse buffer (Millipore), hydrophobic barriers were drawn around each brain section, and slides were placed in a humid chamber for incubation with Blocking Reagent (Millipore) for 20 min at 37° C. Positive and negative controls were run to detect autofluorescence and any non-specific binding. Primary antibodies were diluted in Antibody Diluent (Dako), and a volume of 100 μl was pipetted on each tissue section for a 48 h incubation at 4° C. in a humid chamber. Sections were then rinsed and incubated in appropriate secondary antibody at a 1:200 dilution for 20 min. After secondary antibody incubation, sections were rinsed with Wash Buffer (Millipore) and then distilled water, and mounted with Light Diagnostics Mounting Fluid (Millipore). Sections were kept in the dark after secondary antibody incubation and immediately analyzed by confocal microscopy on a Zeiss LSM-410 Invert Laser Scan Microscope (Carl Zeiss MicroImaging, Thornwood, N.Y.). Yellow color in the merged images from green channel (neuropeptides) and red channel (BMP family members) was considered as colocalization.

Results show that BMPs colocalize with neuropeptides in the hypothalamus.

Accordingly, these data show mRNA expression of several type I and type II BMP receptors thought to mediate the effects of BMP7 (ALK2/ACVR1, ALK3/BMPR1a, ALK6/BMPR1b, BMPR2, ACVR1a, ACVR2a). Additionally, high hypothalamic mRNA expression of BMP2, 4, 6, and 7 is shown. By LacZ staining the presence of BMP7 in the hypothalamus, and by in situ hybridization the presence of BMPR2 in the hypothalamus is reported. The colocalization of BMP receptors and BMP7 ligand with neuropeptides in the arcuate nucleus of the hypothalamus further suggests that BMP7 signaling may be involved with appetite and energy regulation in those neurons.

### Example 2

### Diet Increases BMP Expression in the Hypothalamus

BMP7 expression was assessed using quantitative PCR (qPCR) in the arcuate nucleus of the hypothalamus (ARC, which contains the first-order neuropeptide-containing neurons) and the second-order neurons of the hypothalamus in male C57BL/6 mice fed a low fat (10%) or high fat (60%) diet for 24 hours, 7 days, and 14 days.

The two hypothalamic regions were dissected separately prior to RNA-extraction and cDNA synthesis to distinguish between expression in the ARC first-order neurons which receive signals from the circulation, and expression in the second-order neurons which receive neuropeptide signals from the ARC.

24 hour, 7 day, and 14 day time points were chosen to represent acute high fat feeding without being confounded by circadian rhythms (24 hr time-point), and short-term high-fat feeding (7 days, when it has been shown that body weights of these mice diverge on a high fat diet, and arcuate nucleus leptin resistance has begun; and 14 days when body weights have further diverged). While all the ligands, receptors and cofactors we measured were expressed at high levels in both areas of the hypothalamus, BMP5, BMP8a, and BMP8b were all expressed at levels below threshold.

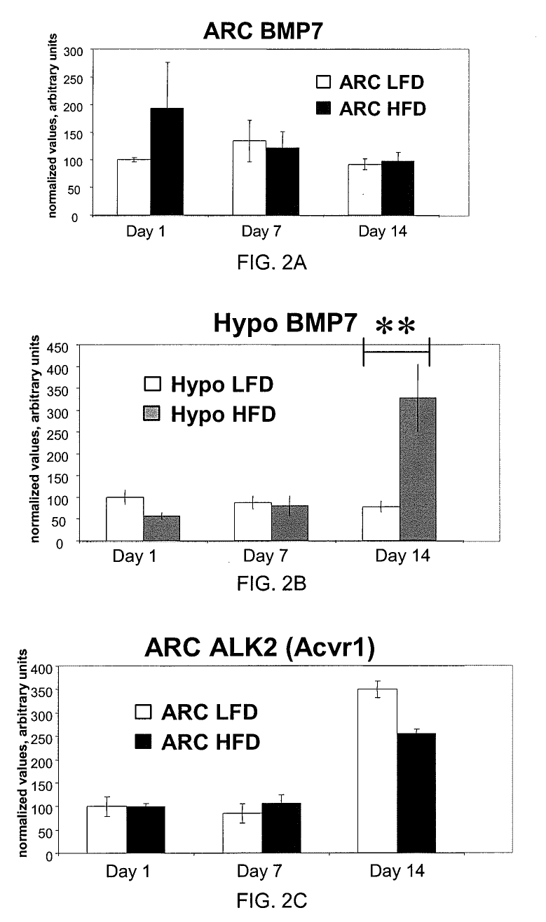

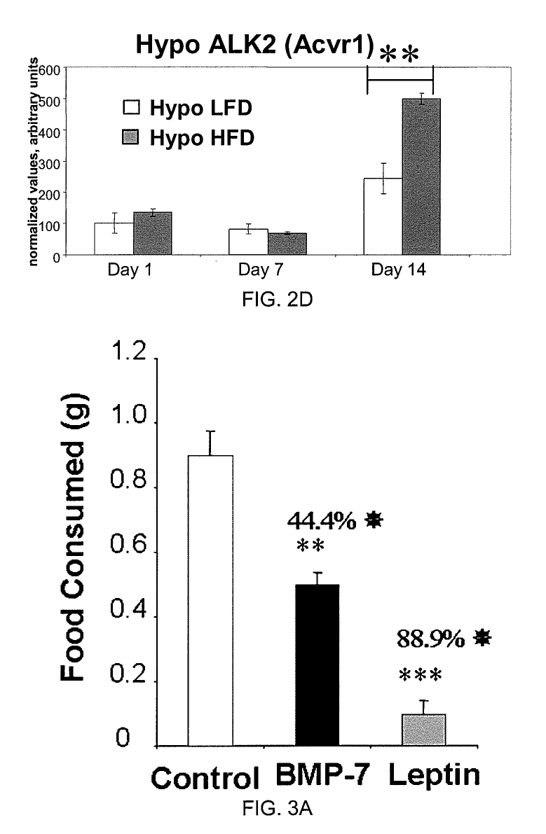

As shown in FIGS. 2B and 2D, expression of both BMP7 and the type I receptor Acvr1 (ALK2) were increased at 14 days in the second-order neurons (Hypo) in mice fed a high fat diet, but not in the arcuate nucleus (ARC) (FIGS. 2A and 2C). All other ligands, receptors and co-factors measured (BMP2, 4, 6, BMPR1a/ALK3, BMPR1b/ALK6, BMPR2, Acvr2a, Avcr2b, and RGMA) were unchanged in both arcuate and second-order neurons at all time points.

These data indicate that neuronal BMP7 expression is affected by high fat food intake.

These data show that at 14 days of a high fat diet, when arcuate nucleus leptin resistance and weight gain have begun in C57BL6 mice, there is an increase in BMP7 and one of its receptors, ALK2, in second-order hypothalamic nuclei. ALK2 is a high-affinity BMP7 receptor (Macias-Silva et al., J. Biol. Chem., 273:25628-25636 (1998)). These data suggest activation of a potential compensatory mechanism to overcome diet-induced weight gain. Similar changes are also seen for the leptin system, with leptin expression increased in adipose tissue of obese mice, leading to increased circulating levels which act on anorexigenic neurons in the hypothalamus. With long-term high fat diet this leptin response becomes desensitized and leptin-responsiveness is lost. However, diet-induced obese mice remain responsive to anorexigenic effects of BDNF and CNTF, and in our obese mice BMP7 responsiveness is maintained. Given the changes in BMP7 and ALK2 in second-order hypothalamic neurons and the known leptin-resistance occurring in the arcuate nucleus after a short-term high fat diet, the response of DIO mice to exogenous BMP7 treatment appears to be mediated via these second-order neurons. This observation is supported by the subsequent examples, which show that BMP7 treatment leads to an increase in phosphor-p70S6K in the PVN (one of the hypothalamic nuclei containing second-order neurons).

### Example 3

### Intracerebroventricular Administration of BMP7 Regulates Neuropeptide Expression

The effect of BMP7 on feeding behavior was assessed administering BMP7 or leptin (a strong appetite suppressor), as a control, intracerebroventricularly (ICV) to the lateral ventricle of C57BL6/J mice on a chow diet.

ICV injections were performed as follows. Mice were intraperitoneally anesthetized with 15 mL/kg body weight of a 2.5% solution of 2:1 (weight:weight) mixture of 2,2,2-tribromoethanol and tertiary amyl alcohol. Mice were then placed in a stereotactic device, and a 26-gauge guide cannula (Plastics One Inc., Roanoke, Va.) was inserted into the right lateral cerebral ventricle (1.0 mm posterior, 1.0 mm lateral, and 2.0 mm ventral to the bregma). To prevent the cannula from being blocked by blood clots, a dummy stylet cannula was inserted into each cannula until used. Animals were allowed to recover for 1 week after the operation.

In single ICV injection experiments, cannulated mice were fasted 4 hours before the onset of the dark cycle. Mice then received an ICV injection of BMP7 (1.7 μg in 1.8 μL) or vehicle buffer through the cannula with a Hamilton microsyringe 1-2 hours before onset of the dark cycle. 10% fat diet pellets were returned to the cages immediately after injection.

In double ICV injection experiments, food was removed 6 hours before onset of the dark cycle. Rapamycin or DMSO was injected 2 hours before the administration of BMP7 (2 μg in 1 μL) or buffer, which was injected 1-2 hours before the onset of the dark cycle. Food intake was measured at the indicated time points after injection.

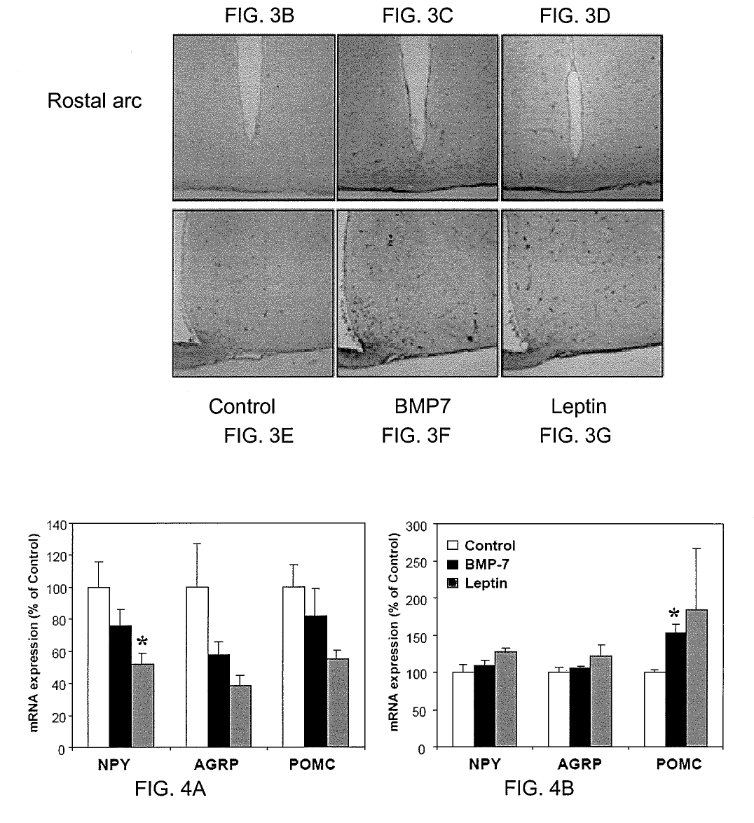

For the data shown in FIG. 3A, injections were performed within 2 hours of the start of the dark cycle—when mice consume most of their food). Food intake was then monitored for 4 hours.

As shown in FIG. 3A, at the 4 hour time point, BMP7 treated mice consumed 44.4% less food compared to vehicle treated mice (number (n)=7). By comparison, leptin evoked about an 89% decrease in food intake. Furthermore, data suggest that the observed BMP7-mediated anorectic effect is acute.

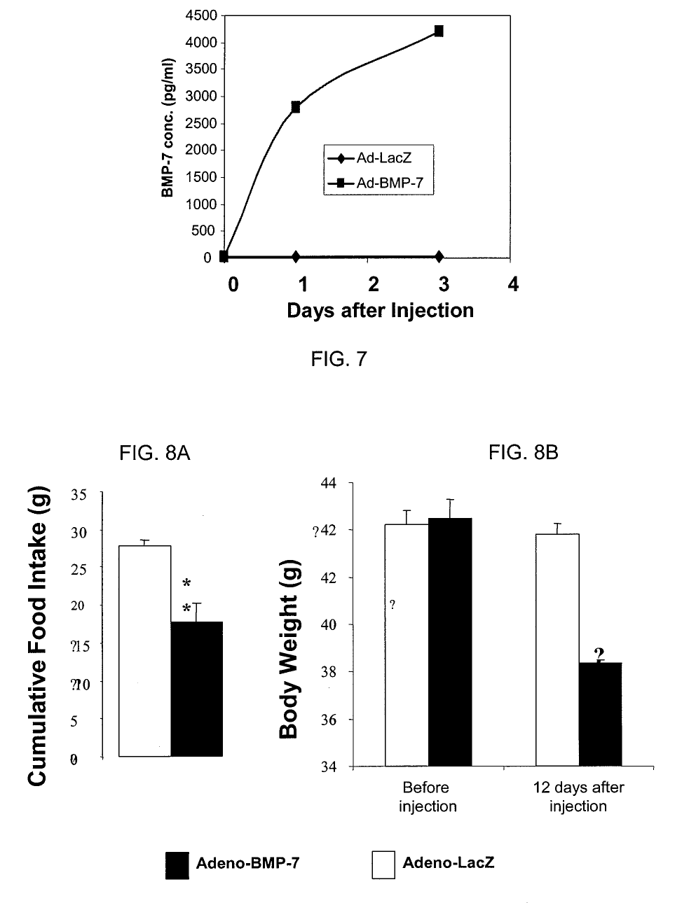

A 2-fold increase in circulating BMP7 levels were detected at 4 hrs post ICV (see FIG. 7). This observation supports that BMP7 can cross the blood brain barrier.

As shown in FIGS. 3B-3G, increased c-FOS IHC was detected 60 minutes after ICV injection of BMP7 and leptin in both the rostral and caudal arcuate nucleus, an area of the hypothalamus containing anorexigenic POMC neurons which supply αMSH to receptors in second-order hypothalamic neurons. c-FOS is an immediate early gene and marker of neuronal activation, accordingly, this observation supports that BMP7, like leptin, promotes increased neural activity.

It has been shown that leptin up-regulates POMC (anorexigenic) and has the opposite effect on NPY and AgRP (orexigenic) expression (Arora and Anubhuti, Neuropeptides, 40:375-401 (2006)). The affect of i.c.v. BMP7 treatment on the same neuropeptides was investigated to characterize the mechanism by which BMP7 promotes the anorectic effect shown in FIG. 3A and elsewhere herein.

ICV injections were performed as described above. Samples of whole hypothalamus were obtained at 2 and 4 hours post ICV. RT-PCR was then performed as follows. Total RNA was isolated with QIAzol lysis reagent (Qiagen, Valencia, Calif.) and purified by RNeasy Mini columns (Qiagen) following the manufacture's instructions. cDNA was prepared from 1 μg of RNA using the Advantage RT-PCR kit (BD Biosciences, Palo Alto, Calif.) according to manufacturer's instructions and diluted to a final volume of 250 μl. 5 μl of diluted cDNA was used in a 20 μl PCR reaction with SYBR Green Master Mix (Applied Biosystems, Foster City, Calif.) and primers at a concentration of 300 nM each. PCR reactions were run in duplicate for each sample and quantitated in the ABI Prism 7000 Sequence Detection System (Applied Biosystems). Data were expressed as arbitrary units after normalization to levels of expression of internal controls Acidic Ribosomal Phosphoprotein P0 (Arbp, 36B4) for each sample. The primers used are shown in Table 1.

TABLE 1

| | | | SEQ |
|---|---|---|---|
| Gene | | | ID |
| Name | Sense Primer | Antisense Primer | NO: |
| NPY | TATCTCTGCTCGTGTGTT | ATTGATGTAGTGTCGCA | 8/9 |
| | TGGGCA | GAGCGGA | |
| POMC | CGGCCCCAGGAACAGCAG | GGGCCCGTCGTCCTTCT | 10/11 |
| | CAGT | CC | |
| AgRP | TTGTGTTCTGCTGTTGGC | AGCAAAAGGCATTGAAG | 11/12 |
| | ACT | AAGC | |
| ALK2 | TGCTAATGATGATGGCTT | TTCACAGTGGTCCTCGT | 13/14 |
| (ACVR1) | TCC | TCC | |

As shown in FIG. 4A, at 2 hours post-ICV, there was a 50% decrease in NPY for leptin-treated mice. POMC and AgRP levels were unchanged. As shown in FIG. 4B, at 4 hours post-treatment there was a 60% upregulation of POMC expression for BMP7 and leptin treatment (the result for leptin did not reach significance (FIG. 4B)). No change was observed for NPY and AgR. These findings are consistent with the acute inhibition of food intake in BMP7 treated mice observed in FIG. 3A.

It was also observed by immunohistochemistry that αMSH, one of the products of POMC-processing, which acts on melanocortin receptors in the hypothalamus to activate appetite-inhibiting pathways, was also increased in the hypothalamus of BMP7 and leptin treated mice (60 minutes after ICV treatment) (FIG. 3e).

Together, these results support that BMP7 suppresses appetite by regulating various neuropeptides in a manner analogous to that of leptin.

### Example 4

### BMP7 Engages Multiple Signaling Pathways in Appetite Regulating Centers in the Hypothalamus

BMPs, including BMP7, activate signaling pathways including the SMAD pathway and the p38-MAPK pathway. Recently BMPs have been found to activate non-canonical signaling pathways including the mTOR and STAT3 pathways (Langenfeld et al., Mol. Cancer. Res., 3:679-684 (2005); Mikami et al., J. Cell. Physiol., 223:123-133 (2010); and Fukuda et al., Mol. Cell. Biol., 27:4931-4937 (2007)), which pathways are reportedly involved in appetite regulation and metabolism in the hypothalamus.

To characterize the neural peptides activated by BMP7, ICV injections were performed as described in Example 3. Samples were then collected as described below at various time points post ICV and Western Blots were performed to detect various phosphorylated proteins, as follows.

Cells were harvested in lysis buffer (50 mM HEPES, 137 mM NaCl, 1 mM MgCl2, 1 mM CaCl2, 10 mM Na2P2O7, 10 mM NaF, 2 mM EDTA, 10% glycerol, 1% Igepal CA-630, 2 mM vanadate, 10 μg/ml of leupeptin, 10 μg/ml of aprotinin, 2 mM phenylmethylsulfonyl fluoride; pH 7.4). Lysates were then centrifuged at 12,000×g for 20 min at 4° C. Protein concentrations were subsequently determined using the Bradford Protein Assay (Bio-Rad Laboratories, Hercules, Calif.). Proteins were then solubilized in Laemmli sample buffer and equal amounts of protein were separated by sodium dodecyl sulfate-polyacrylamide gel electrophoresis before transfer to Immobolin-P membranes. Membranes were blocked overnight at 4° C. and incubated with the indicated antibody for 2 hours at room temperature. Specifically bound primary antibodies were detected with peroxidase-coupled secondary antibody and enhanced chemiluminescence (ECL, Amersham Biosciences, Piscataway, N.J.).

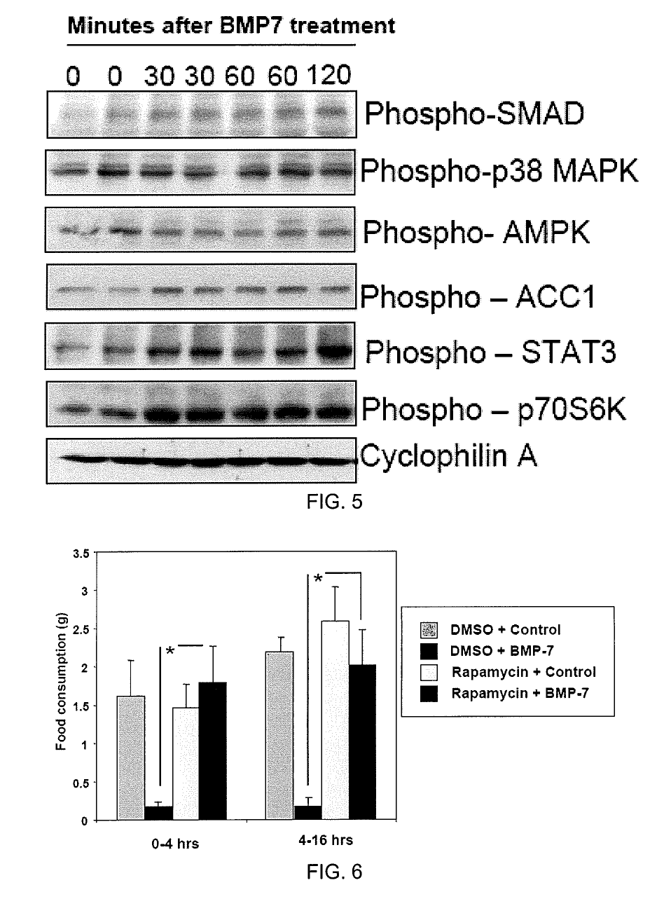

As shown in FIG. 5, ICV BMP7 resulted in phosphorylation of STAT3 and p70S6K. Each of these signaling components are known to be activated/phosphorylated by leptin in the regulation of appetite (STAT3 as part of the JAK2-STAT3 pathway which leads to transcription of POMC; p79S6K as part of the mTOR pathway). ACC1, part of the AMPK metabolic pathway, was also phosphorylated in response to ICV BMP7 treatment. Similar results were also observed in cultured BMP7-treated N25-2 neuronal cells (see FIG. 6). These results were obtained following experimental protocol.

Embryonic Mouse Hypothalamus Cell Line N25/2 (mHypoE-N25/2) (Cellutions biosystems Inc.), p20, were grown in 6 cm dishes in 10% FBS, 0.75% NaHCO3-DMEM-H supplemented with penicillin/streptomycin/fungizone. When cells became approximately 80% confluent, growth medium was replaced with 0.1% BSA, 0.75% NaHCO3-DMEM-H. After 16 hr incubation in this medium, cells were incubated in the presence of 3.3 uM BMP7 (R&D Systems) for 0, 10, 30, and 60 min, or BMP7 solvent buffer (4 mM HCl, 0.1% BSA) for 60 min. Cells were rinsed with cold PBS and immediately frozen on liquid nitrogen and stored at −80° C. until processed. Proteins were extracted with 300 ul of lysis buffer (50 mM HEPES, pH 7.4, 1% Triton X-100, 50 nM Na-pyrophosphate, 100 mM NaF, 10 mM EDTA, 1 mM NaVO4, 10 μg/mL Aprotinin, 10 μg/mL Leupeptin, 2 mM PMSF) per dish. Protein concentration determined using BCA reagent (Pierce). Proteins were separated on 10% SDS-PAGE gels, transferred to PVDF membranes and detected with specific antibodies.

Increased phosphorylation of p70S6K1 in the arcuate nucleus in response to ICV BMP7 treatment was also observed by immunohistochemistry.

To further establish the physiological significance of the mTOR pathway in mediating the anorectic effects of central BMP7, mice were treated ICV with rapamycin, an mTOR inhibitor. This manipulation resulted in a complete restoration of food intake in the BMP7 treated mice (see FIG. 6B), indicating that the mTOR pathway is an important mediator of the central anorectic effects of BMP7 and may be necessary and sufficient for the anorexigenic effects of BMP7. STAT3 inhibitors did not have the same effect.

These observations support that BMP engages multiple signaling pathways in the hypothalamus, and that the mTOR pathway may be important in mediating the anorectic effects of BMP7.

### Example 6

### BMP7 Reduces Appetite and Promotes Weight Loss in Diet-Induced Obesity (DIO) Mice

As shown in Example 3, ICV administered BMP7 promotes acute anorexia. Accordingly, investigations into the long-term effects of BMP7 on body weight regulation using the well-established DIO mouse model, which closely resembles obesity in humans (Augustine and Rossi, Anat. Rec., 257:64-72 (1999)), were performed.

ICV injections were performed as described in Example 3. Where indicated, adenoviral BMP7 was also used, as follows.

Adenoviruses were amplified in HEK293 cells as previously described (Ueki et al., 2004). Prior to in vivo use, all adenoviruses were purified on a cesium chloride gradient and dialyzed into PBS plus 10% glycerol. Four and twelve-week-old male C57BL/6 mice were injected via tail vein with a adenoviral dose 5×10⁸ viral particle/g body weight as described previously (Laustsen et al., Genes Dev., 16:3212-3222 (2002)). Mice were sacrificed at 15 days after injection. Intrerscapular brown fat and epididymal white fat were excised and weighted. Half of the tissue was fixed in 10% formalin and proceed for histological analysis. The other half of the tissue was subjected for RNA extraction and Q-RT-PCR analysis.

Metabolic measurements were taken according to the following protocols.

Metabolic rates were measured by indirect calorimetry in mice 7-10 days after adenoviral injection by using the Comprehensive Lab Animal Monitoring System (CLAMS, Columbus Instruments, Columbus, Ohio). Mice were maintained at 24° C. under a 12-h light/dark cycle. Food and water were available ad libitum. Mice were acclimatized to individual cages for 24 h before recording, and then underwent 24 h of monitoring.

Heat production (energy expenditure) was calculated using the following equations:

Heat [kcal/hr]=(3.815+1.232*(VCO2/VO2))*VO2 [liter/kg/hr]*body weight [kg]

To test the potential role of BMP7 in the control of energy balance and evaluate its potential function in the treatment of obesity, adenoviral-mediated expression of BMP7 was used to increase BMP7 levels in obese mice induced by high fat diet (DIO). Adenoviruses expressing BMP7 or LacZ control were obtained from Dr. Tong-Chuan He at The University of Chicago Medical Center. Eight week-old male C57BL/6 mice (Taconic, Germantown, N.Y.) were fed a high fat diet (containing 44.9% fat; D12451, Research Diets, New Brunswick, N.J.) for 3 months to induce obesity. At this time, the mean mouse weight of the high fat diet mice was 44 g. Half of these obese mice received via tail vein a single injection of 5×10⁸ plaque-forming units BMP7 adenovirus per gram of body weight (n=8 per group) and half received a LacZ-expressing adenovirus as a negative control. Such treatment has been shown to yield high levels of expression of the transgene in the liver (Tseng et al., Nature 454:1000-1004 (2008)). Since the construct contained a signal peptide, BMP7 was expressed by hepatocytes and secreted into circulation (the adenovirus is tropic for the liver, and BMP7 is released to the blood stream) as a secreted protein. Indeed, serum BMP7 levels were elevated to about 15-fold in mice receiving adenoviruses expressing BMP7, but not the lac Z control. At day 3 post infection, serum BMP levels, as measured by ELISA, were 4 ng/ml and at 16 days 1.3 ng/ml, while endogenous BMP7 remained undetected using the same assay (see FIG. 7). ELISA was performed as follows. Blood was collected at day 1 and day 3 after adenoviral injection and at the time of sacrifice. Plasma BMP7 levels were determined by ELISA assay using the DuoSet ELISA Development kit purchased from R&D Systems following the manufacture's instructions. Concentrations were calculated using a standard curve generated by rhBMP7 standards included in the kit.

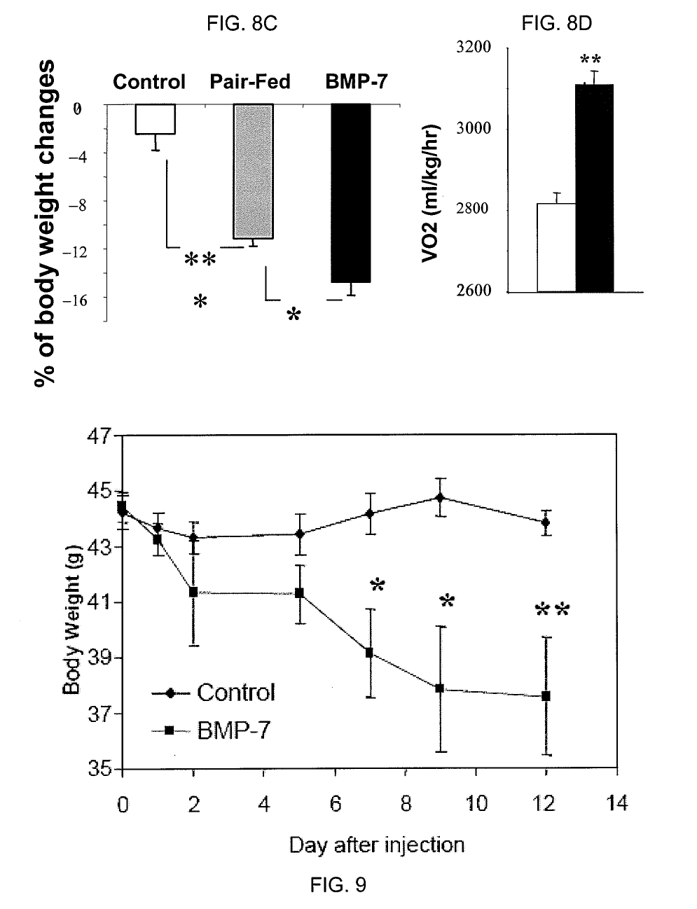

BMP7 caused a progressive and significant weight loss as compared to the control group (FIGS. 8B-8C). Twelve days after injection, BMP7 reduced body weight of DIO mice by 14% with no apparent toxicity. The anti-obesity effect of BMP7 is mediated by a decrease in food intake (FIG. 8A) as well as a significant increase in energy expenditure as measured by oxygen consumption (FIG. 8D).

As shown in FIG. 9, BMP7 treated mice lost 15% of their initial body during the 12 days following adenovirus injection, in contrast to 2% seen in LacZ-treated. At sacrifice, ALT serum levels, a marker of adenovirus mediated liver injury, were indistinguishable between the two treatment groups, confirming the specificity of the BMP7 effects.

Two factors appear to contribute to leanness in this mouse model: namely, increased energy expenditure and reduced caloric intake, as indicated in Example 3. Indeed, during this study, BMP7-adenovirus treated mice had an increased oxygen consumption, indicative of energy expenditure (see FIG. 8D) and consumed 30% fewer calories overall compared to the mice treated with control adenovirus (see FIG. 8A). The appetite-reducing effect of peripheral administration of BMP7 (directly leading to increased circulating levels), further suggests that BMP7 is able to cross the blood brain barrier, as decreased food intake is mediated by brain regions such as the hypothalamus.

The inclusion of an additional group of mice in our experimental design, which were treated with control adenovirus but fed the same amount of food consumed by the BMP7 treated mice the previous day (designated pair-fed group) revealed that increased energy expenditure accounted for only one third of the weight loss in BMP7 treated mice (see FIG. 8C). The rest of weight loss in the BMP7 treated obese mice could be attributed to their reduced food consumption.

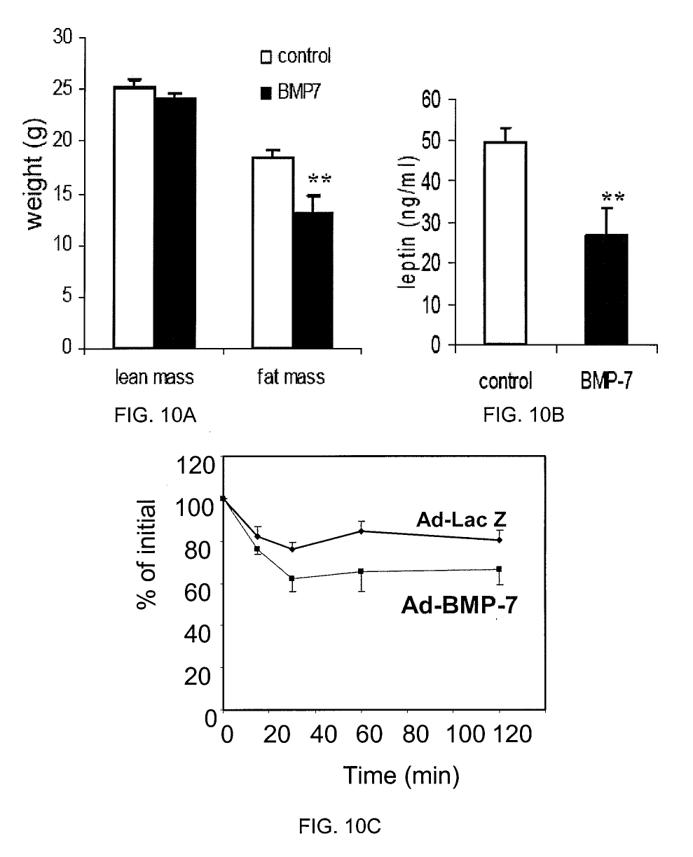

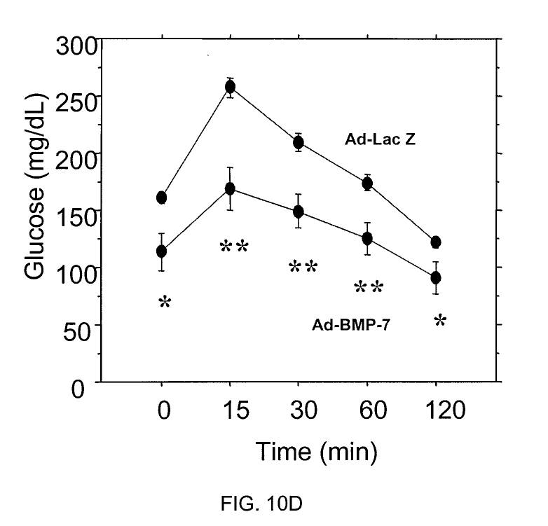

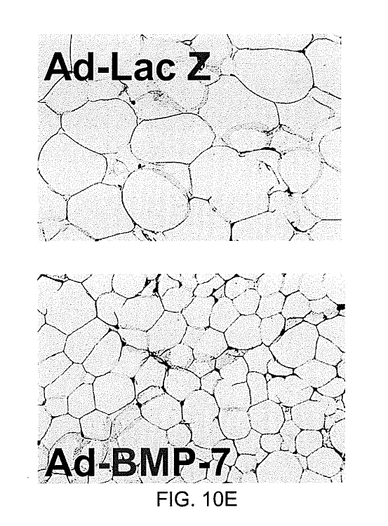

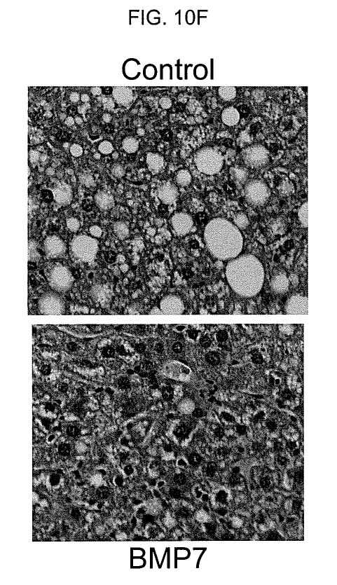

As shown in FIG. 10A, the BMP7 associated leanness in DIO mice was due to loss of fat, but not loss of lean mass. Consistent with this observation, as shown in FIG. 10B, BMP7 treated mice had significantly lower serum leptin levels (p<0.0001) than control. As shown in FIGS. 12B and 12D, BMP7 induced weight loss in DIO mice was also associated with improvements in additional parameters of the metabolic syndrome, namely insulin resistance and development of hepatic steatosis.

Accordingly these results support that BMP7 promotes decreased appetite and weight loss in an art-recognized animal model of obesity.

### Example 7

### BMP7 Reduces Appetite and Promotes Weight Loss in Leptin Deficient (Ob/Ob) Mice

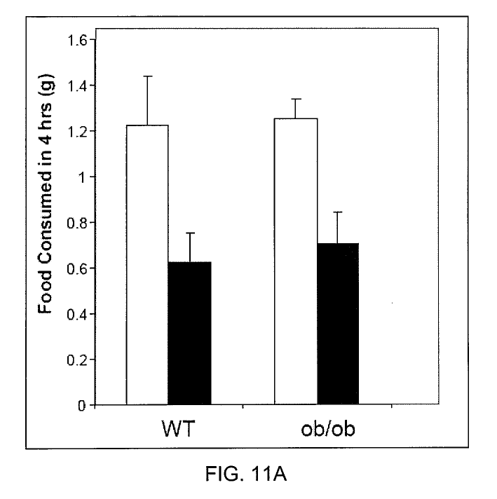

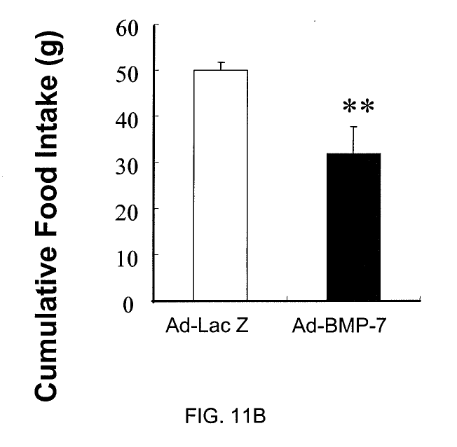

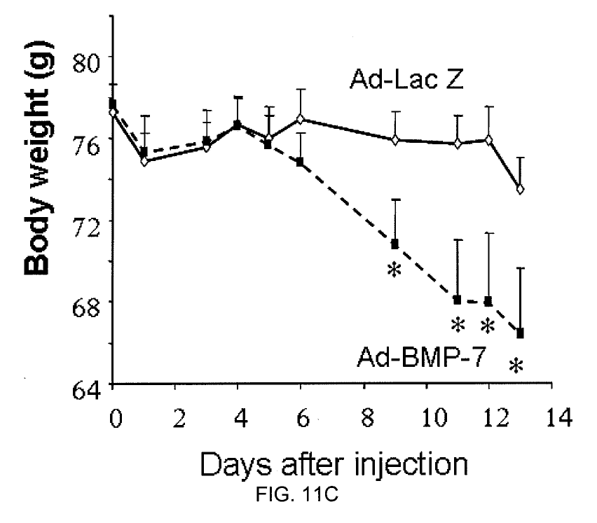

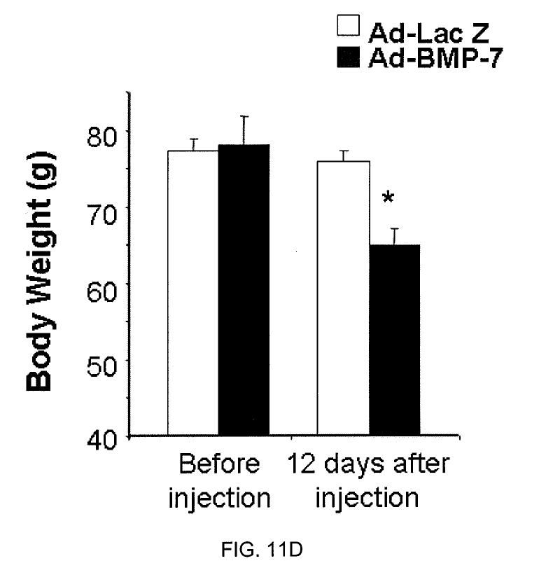

The methods described in Example 6 were applied to the Ob/Ob mouse animal model to investigate whether the effects of BMP7 on appetite and energy balance are leptin-independent (see FIG. 11).

As shown in FIGS. 11B-11D, systemic treatment of ob/ob mice with a BMP7 producing adenovirus resulted in profound weight loss (11C) and a significant reduction in food intake (11B and 11D). Specifically, up to day 12 following adenoviral injection, ob/ob mice treated with BMP7 had consumed 30% less food than controls (FIG. 11B). During the same period, a 13% reduction compare to mouse initial body weight was apparent (FIG. 11C).

BMP7 was also administered ICV. As shown in FIG. 11A, a decrease in food intake similar to that seen in wild-type controls.

Taken together, these results support that BMP7 does not require leptin to mediate its appetite and weight reducing effects.

### Example 8

### BMP7 Treatment Reduces Insulin Resistance, Promotes Glucose Sensitivity and Protects Against Indications of Obesity

Glucose and insulin tolerance tests were performed as follows. Animals were subjected to a 4-15 hour fasting period (with free access to water), whereafter either 2 g/kg BW of glucose or 0.75 iU/kg BW of insulin was injected intraperitoneally. Blood glucose concentrations were determined by the tail nick procedure. Blood was collected at 10 minutes pre-injection and 15, 30, 60 and 120 minutes post injection. Mice were fed immediately after the last blood sample was collected. In order to reduce the numbers of animals used in experiments, these two tests were performed sequentially with a 1-2 weeks interval between tests.

As shown in FIG. 10C, BMP7 improves insulin sensitivity compared to control (LacZ). BMP7 also improves glucose sensitivity in treated animals (FIG. 10D). As a result, BMP7 also reduces fat stores (FIG. 10E) and reduces hepatic steatosis (FIG. 10F).

The data presented in the above examples support that BMP7 is a neurotrophic factors involved in both neuronal development and adult body weight homeostasis via hypothalamic targets and that BMP7's action is leptin-independent.

These data show that BMP7 can act independently of leptin, as the leptin deficient ob/ob mice are still responsive to the weight-lowering effects of BMP7. BMP7 is unique, with respect to other anorexigenic neurotrophic facts, such as BNDF, which is leptin-dependent (Komori et al., Neuroscience, 139:1107-1115 (2006)). BMP7's leptin-independency is similar to that of CNTF as both exert anorexigenic effects in the hypothalamus independently of leptin and in leptin-resistant states such as diet-induced obesity.

This and previous data support that BMP7 has a two-pronged effect to reduce adiposity. Specifically, BMP7 acts in the periphery to stimulate brown fat thermogenesis and energy expenditure ((Tseng et al., Nature, 454:1000-1004 (2008)), and via the hypothalamus to reduce food intake. Overall, the appetite-regulatory effects of BMP7 are suggestive of a family of neurotrophic factors which not only play an important role in neuronal development and plasticity, but also in regulating appetite via actions on the hypothalamus. BMP7s ability to activate hypothalamic mTOR signaling appears to be required for its appetite-reducing role.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of suppressing appetite for food intake in a subject, the method comprising increasing bone morphogenic protein 7 (BMP7) expression, activity, or signaling in the central nervous system of the subject, the method comprising administering one or more BMP7 agents to the subject.

2. The method of claim 1, further comprising selecting the subject on the basis that the subject is in need of appetite suppression.

3. The method of claim 1, further comprising:
evaluating food intake at a first time to establish a first value;
evaluating food intake at a second time after administration of one or more doses of the BMP7 agent to establish a second value;
comparing the first value and the second value to detect an effect of the BMP7 agent on appetite,
wherein a decrease in food intake from the first value to the second value indicates the effect of the BMP7 on appetite in the subject.

4. The method of claim 3, further comprising adjusting the dose of the BMP7 agent to optimize the effect on appetite in the subject.

5. The method of claim 1, wherein the BMP7 agent is administered systemically.

6. The method of claim 1, wherein the one or more BMP7 agents comprises a BMP7 polypeptide.

7. The method of claim 6, wherein the BMP7 polypeptide comprises a polypeptide with at least 95% identity to amino acids 293-431 of SEQ ID NO:5.

8. The method of claim 1, wherein the BMP7 agent comprises a BMP7 peptide or peptide mimetic.

9. The method of claim 1, wherein the one or more BMP7 agents comprises a nucleic acid molecule encoding a BMP7 polypeptide.

10. The method of claim 9, wherein the nucleic acid molecule encodes a BMP7 polypeptide with at least 95% identity to amino acids 293-431 of SEQ ID NO:5.

11. The method of claim 1, comprising selecting a subject who has a body mass index (BMI) of at least 25.

12. The method of claim 11, wherein the subject has a BMI of at least 30.

13. The method of claim 1, comprising selecting a subject has a daily food intake above a selected level.
